(19) United States
(12) Patent Application Publication
(10) Pub. No.: US 2012/0035169 A1
(43) Pub. Date: Feb. 9, 2012

(54) AEROSOL FORMULATION FOR THE INHALATION OF BETA-AGONISTS

(75) Inventor: Michael AVEN, Mainz (DE)

(73) Assignee: BOEHRINGER INGELHEIM INTERNATIONAL GMBH, Ingelheim am Rhein (DE)

(21) Appl. No.: 13/232,357

(22) Filed: Sep. 14, 2011

Related U.S. Application Data
(63) Continuation of application No. 12/133,066, filed on Jun. 4, 2008, now abandoned.
Application No. 12/133,066 is a continuation of application No. 11/125,756, filed on May 10, 2005, now abandoned.
(60) Provisional application No. 60/578,541, filed on Jun. 10, 2004.

(30) Foreign Application Priority Data
May 14, 2004 (DE) 102004024452

(51) Int. Cl. A61K 31/538; A61P 11/00

(52) U.S. Cl. 514/230.5

(57) ABSTRACT

The present invention relates to a propellant-free aerosol formulation which [contains] one or more compounds of general formula 1

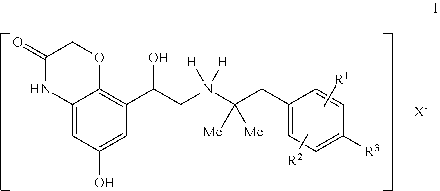

wherein the groups R¹, R², R³ and X⁻ may have the meanings given in the claims and specification, for inhalation.

This application is a continuation of U.S. application Ser. No. 12/133,066 filed Jun. 4, 2008, which is a continuation of U.S. application Ser. No. 11/125,756 filed May 10, 2005, which claims priority benefit under 35 USC 119(e) to U.S. Provisional Application 60/578,541, filed Jun. 10, 2004 and under 35 USC 119(a) to German Application DE 10 2004 024 452 filed May 14, 2004.

The present invention relates to a propellant-free aerosol formulation which [contains] one or more compounds of general formula 1

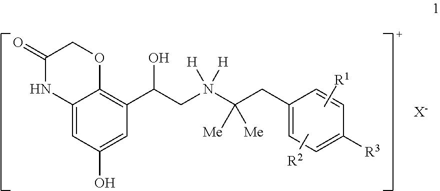

wherein the groups R¹, R², R³ and X⁻ may have the meanings given in the claims and in the specification, for inhalation.

### BACKGROUND TO THE INVENTION

Betamimetics (β-adrenergic substances) are known from the prior art. For example reference may be made in this respect to the disclosure of U.S. Pat. No. 4,460,581, which proposes betamimetics for the treatment of a range of diseases.

For drug treatment of diseases it is often desirable to prepare medicaments with a longer duration of activity. As a rule, this ensures that the concentration of the active substance in the body needed to achieve the therapeutic effect is guaranteed for a longer period without the need to re-administer the drug at frequent intervals. Moreover, giving an active substance at longer time intervals contributes to the well-being of the patient to a high degree. It is particularly desirable to prepare a pharmaceutical composition which can be used therapeutically by administration once a day (single dose). The use of a drug once a day has the advantage that the patient can become accustomed relatively quickly to regularly taking the drug at certain times of the day.

The aim of the present invention is therefore to provide medicament formulations for inhalation which on the one hand confer a therapeutic benefit in the treatment of respiratory complaints and are also characterised by a longer duration of activity and can thus be used to prepare pharmaceutical compositions with a longer duration of activity.

### DETAILED DESCRIPTION OF THE INVENTION

To solve the problems mentioned above the present invention proposes the following medicament formulations.

The medicament formulations according to the invention are propellant-free medicament formulations, containing as sole active substance one or more compounds of general formula 1

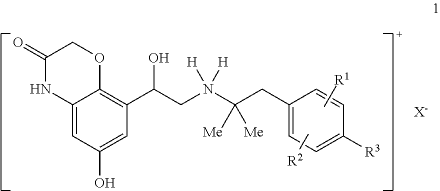

wherein
R¹ denotes hydrogen, C₁-C₄-alkyl, C₁-C₄-alkoxy or halogen;
R² denotes hydrogen, C₁-C₄-alkyl, C₁-C₄-alkoxy or halogen;
R³ denotes hydrogen, C₁-C₄-alkyl, C₁-C₄-alkoxy, halogen, OH, —O—C₁-C₄-alkylene-COOH or —O—C₁-C₄-alkylene-COO—C₁-C₄-alkyl,
X⁻ denotes an anion with a single negative charge, preferably an anion with a single negative charge selected from the group consisting of chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
   optionally in the form of their tautomers, enantiomers, mixtures of enantiomers, racemates or solvates,
   at least one pharmacologically acceptable acid, optionally other pharmacologically acceptable excipients and/or complexing agents and, as solvent, water, ethanol or a mixture of water and ethanol.

Preferred are pharmaceutical formulations which contain compounds of general formula 1, wherein
R¹ denotes hydrogen, methyl, ethyl, fluorine or chlorine;
R² denotes hydrogen, methyl, ethyl, fluorine or chlorine;
R³ denotes hydrogen, methyl, ethyl, propyl, OH, methoxy, ethoxy, fluorine, chlorine, bromine, —O—CH₂—COOH, —O—CH₂—COOmethyl or —O—CH₂—COOethyl, —O—CH₂—CH₂COOH, —O—CH₂—CH₂COOmethyl or —O—CH₂—CH₂COOethyl, —O—CH₂—CH₂—CH₂COOH, —O—CH₂—CH₂—CH₂COOmethyl or —O—CH₂—CH₂—CH₂COOethyl;
X⁻ denotes an anion with a single negative charge, preferably an anion with a single negative charge selected from the group consisting of chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
   optionally in the form of their tautomers, enantiomers, mixtures of enantiomers, racemates or solvates.

Preferred are pharmaceutical formulations which contain compounds of general formula 1 wherein
R¹ denotes hydrogen or methyl, preferably hydrogen;
R² denotes hydrogen or methyl, preferably hydrogen;
R³ denotes methyl, OH, methoxy, fluorine, chlorine, bromine, —O—CH₂—COOH or —O—CH₂—COOethyl;
   X⁻ denotes an anion with a single negative charge selected from the group consisting of chloride, bromide, sulphate, methanesulphonate, maleate, acetate, benzoate, citrate, salicylate, trifluoroacetate, fumarate, tartrate and succinate;
   optionally in the form of their tautomers, enantiomers, mixtures of enantiomers, racemates or solvates.

Also preferred are pharmaceutical formulations which contain compounds of general formula 1 wherein
R³ denotes methoxy, ethoxy, fluorine, chlorine, bromine, —O—CH₂—COOH, —O—CH₂—COOmethyl or —O—CH₂—COOethyl;
   and R¹, R² and X⁻ may have the meanings given above,
   optionally in the form of their tautomers, enantiomers, mixtures of enantiomers, racemates or solvates.

Also preferred are pharmaceutical formulations which contain compounds of general formula 1 wherein
R¹ and R² denote hydrogen;
R³ denotes OH, fluorine, chlorine, methoxy, ethoxy, —O—CH₂—COOH, preferably OH, fluorine, chlorine, ethoxy or methoxy, and
   X⁻ may have one of the meanings given above,
   optionally in the form of their tautomers, enantiomers, mixtures of enantiomers, racemates or solvates.

Also preferred are pharmaceutical formulations which contain compounds of general formula 1 which are selected from the following, wherein HX denotes an acid, wherein X⁻ may have one of the meanings given above:
6-hydroxy-8-{1-hydroxy-2-[2-(4-methoxy-phenyl)-1,1-dimethyl-ethylamino]-ethyl}-4H-benzo[1,4]oxazin-3-one;
6-hydroxy-8-{1-hydroxy-2-[2-(4-phenoxyethyl-acetate)-1,1-dimethyl-ethylamino]-ethyl}-4H-benzo[1,4]oxazin-3-one;
6-hydroxy-8-{1-hydroxy-2-[2-(4-phenoxy-acetic acid)-1,1-dimethyl-ethylamino]-ethyl}-4H-benzo[1,4]oxazin-3-one;
8-{2-[1,1-dimethyl-2-(2,4,6-trimethylphenyl)-ethylamino]-1-hydroxy-ethyl}-6-hydroxy-4H-benzo[1,4]oxazin-3-one;
6-hydroxy-8-{1-hydroxy-2-[2-(4-hydroxy-phenyl)-1,1-dimethyl-ethylamino]-ethyl}-4H-benzo[1,4]oxazin-3-one;
6-hydroxy-8-{1-hydroxy-2-[2-(4-isopropyl-phenyl)-1,1-dimethyl-ethylamino]-ethyl}-4H-benzo[1,4]oxazin-3-one;
8-{2-[2-(4-ethyl-phenyl)-1,1-dimethyl-ethylamino]-1-hydroxy-ethyl}-6-hydroxy-4H-benzo[1,4]oxazin-3-one;
8-{2-[2-(4-fluoro-3-methyl-phenyl)-1,1-dimethyl-ethylamino]-1-hydroxy-ethyl}-6-hydroxy-4H-benzo[1,4]oxazin-3-one;
8-{2-[2-(4-fluoro-2-methyl-phenyl)-1,1-dimethyl-ethylamino]-1-hydroxy-ethyl}-6-hydroxy-4H-benzo[1,4]oxazin-3-one;
8-{2-[2-(2,4-difluoro-phenyl)-1,1-dimethyl-ethylamino]-1-hydroxy-ethyl}-6-hydroxy-4H-benzo[1,4]oxazin-3-one;
8-{2-[2-(3,5-difluoro-phenyl)-1,1-dimethyl-ethylamino]-1-hydroxy-ethyl}-6-hydroxy-4H-benzo[1,4]oxazin-3-one;
8-{2-[2-(4-ethoxy-phenyl)-1,1-dimethyl-ethylamino]-1-hydroxy-ethyl}-6-hydroxy-4H-benzo[1,4]oxazin-3-one;
8-{2-[2-(3,5-dimethyl-phenyl)-1,1-dimethyl-ethylamino]-1-hydroxy-ethyl}-6-hydroxy-4H-benzo[1,4]oxazin-3-one;
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid;
8-{2-[2-(3,4-difluoro-phenyl)-1,1-dimethyl-ethylamino]-1-hydroxy-ethyl}-6-hydroxy-4H-benzo[1,4]oxazin-3-one;
8-{2-[2-(2-chloro-4-fluoro-phenyl)-1,1-dimethyl-ethylamino]-1-hydroxy-ethyl}-6-hydroxy-4H-benzo[1,4]oxazin-3-one;
8-{2-[2-(4-chloro-phenyl)-1,1-dimethyl-ethylamino]-1-hydroxy-ethyl}-6-hydroxy-4H-benzo[1,4]oxazin-3-one;
8-{2-[2-(4-bromo-phenyl)-1,1-dimethyl-ethylamino]-1-hydroxy-ethyl}-6-hydroxy-4H-benzo[1,4]oxazin-3-one;
8-{2-[2-(4-fluoro-phenyl)-1,1-dimethyl-ethylamino]-1-hydroxy-ethyl}-6-hydroxy-4H-benzo[1,4]oxazin-3-one;
8-{2-[2-(4-fluoro-3-methoxy-phenyl)-1,1-dimethyl-ethylamino]-1-hydroxy-ethyl}-6-hydroxy-4H-benzo[1,4]oxazin-3-one;
8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;
8-{2-[2-(4-chloro-2-methyl-phenyl)-1,1-dimethyl-ethylamino]-1-hydroxy-ethyl}-6-hydroxy-4H-benzo[1,4]oxazin-3-one;
8-{2-[2-(4-chloro-3-fluoro-phenyl)-1,1-dimethyl-ethylamino]-1-hydroxy-ethyl}-6-hydroxy-4H-benzo[1,4]oxazin-3-one;
8-{2-[2-(4-chloro-2-fluoro-phenyl)-1,1-dimethyl-ethylamino]-1-hydroxy-ethyl}-6-hydroxy-4H-benzo[1,4]oxazin-3-one;
8-{2-[2-(3-chloro-4-fluoro-phenyl)-1,1-dimethyl-ethylamino]-1-hydroxy-ethyl}-6-hydroxy-4H-benzo[1,4]oxazin-3-one;
8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;
8-{2-[2-(2,5-difluoro-4-methoxy-phenyl)-1,1-dimethyl-ethylamino]-1-hydroxy-ethyl}-6-hydroxy-4H-benzo[1,4]oxazin-3-one;
8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;
8-{2-[2-(3,5-dichloro-phenyl)-1,1-dimethyl-ethylamino]-1-hydroxy-ethyl}-6-hydroxy-4H-benzo[1,4]oxazin-3-one;
8-{2-[2-(4-chloro-3-methyl-phenyl)-1,1-dimethyl-ethylamino]-1-hydroxy-ethyl}-6-hydroxy-4H-benzo[1,4]oxazin-3-one;
8-{2-[2-(3,4,5-trifluoro-phenyl)-1,1-dimethyl-ethylamino]-1-hydroxy-ethyl}-6-hydroxy-4H-benzo[1,4]oxazin-3-one;
8-{2-[2-(3-methyl-phenyl)-1,1-dimethyl-ethylamino]-1-hydroxy-ethyl}-6-hydroxy-4H-benzo[1,4]oxazin-3-one and
8-{2-[2-(3,4-dichloro-phenyl)-1,1-dimethyl-ethylamino]-1-hydroxy-ethyl}-6-hydroxy-4H-benzo[1,4]oxazin-3-one,
   in each case in the form of an acid addition salt with an acid HX, wherein X⁻ may have one of the meanings given above, as well as optionally in the form of their tautomers, enantiomers, mixtures of enantiomers, racemates or solvates.

Unless otherwise stated, the alkyl groups are straight-chained or branched alkyl groups having 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the alkylene groups are branched and unbranched double-bonded alkyl bridges having 1 to 4 carbon atoms. The following are mentioned by way of example: methylene, ethylene, n-propylene or n-butylene.

Unless otherwise stated, the term alkyloxy groups (or —O-alkyl or alkoxy groups) denotes branched and unbranched alkyl groups having 1 to 4 carbon atoms which are linked via an oxygen atom. Examples of these include: methyloxy, ethyloxy, propyloxy or butyloxy. The abbreviations MeO—, EtO—, PropO— or BuO— are used in some cases to denote the groups methyloxy, ethyloxy, propyloxy or butyloxy. Unless otherwise stated, the definitions propyloxy and butyloxy include all possible isomeric forms of the groups in question. Thus, for example, propyloxy includes n-propyloxy and iso-propyloxy, butyloxy includes iso-butyloxy, sec.butyloxy and tert.-butyloxy, etc. In some cases, within the scope of the present invention, the term alkoxy is used instead of the term alkyloxy. Accordingly, the terms methoxy, ethoxy, propoxy or butoxy may also be used to denote the groups methyloxy, ethyloxy, propyloxy or butyloxy.

Halogen within the scope of the present invention denotes fluorine, chlorine, bromine or iodine. Unless stated otherwise, fluorine, chlorine and bromine are the preferred halogens.

The medicament formulations according to the invention contain as solvent pure water, pure ethanol or mixtures of ethanol and water. If ethanol-water mixtures are used, the percentage by mass of ethanol in these mixtures is preferably in the range between 5 and 99% ethanol, particularly preferably in the range from 10 to 96% ethanol. Most particularly preferred medicament formulations for the purposes of the present invention contain as solvent pure water, pure ethanol or ethanol-water mixtures containing between 50 and 92%, particularly preferably between 69 and 91% ethanol.

If desired, other co-solvents may be used in addition to ethanol and water. Preferably, however, no other solvent is used according to the invention.

The compounds according to the invention may be prepared analogously to methods already known from the prior art. Suitable methods of preparation are known for example from U.S. Pat. No. 4,460,581, to the entire contents of which reference is made at this point.

The compounds of formula 1 may optionally be present in the medicament formulations according to the invention in the form of their tautomers. By tautomerism is meant the occurrence of isomeric compounds which are formed by the shifting of σ or π bonds and may be present in equilibrium. Examples of possible tautomeric forms of the compounds of formula 1 are

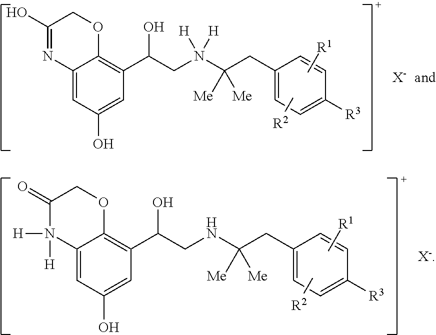

In another aspect the present invention relates to pharmaceutical formulations which contain the above-mentioned compounds of formula 1 in the form of the individual optical isomers, mixtures of individual enantiomers or racemates. Particularly preferred are medicament formulations which contain the above-mentioned compounds of formula 1 in the form of the enantiomerically pure compounds, while the R-enantiomers of the compounds of formula 1 according to the invention are of particular importance. These R-enantiomers may be represented by general formula R-1

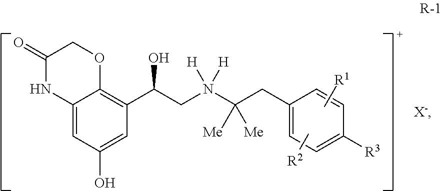

wherein the groups R¹, R², R³ and X⁻ may have the meanings given above.

In another aspect the present invention relates to the use of the pharmaceutical formulations according to the invention for preparing a pharmaceutical composition for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament formulations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma or COPD.

It is also preferable to use the medicament formulations according to the invention for preparing a pharmaceutical composition for the treatment of pulmonary emphysema which has its origins in COPD (chronic obstructive pulmonary disease) or αl-proteinase inhibitor deficiency.

It is also preferable to use the medicament formulations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament formulations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic scleroderma or sarcoidosis, granulomatoses, such as for example Boeck's disease, idiopathic interstitial pneumonia or idiopathic pulmonary fibrosis (IPF).

It is also preferable to use the medicament formulations according to the invention for preparing a pharmaceutical composition for the treatment of cystic fibrosis or mucoviscidosis.

It is also preferable to use the medicament formulations according to the invention for preparing a pharmaceutical composition for the treatment of bronchitis, such as for example bronchitis caused by bacterial or viral infection, allergic bronchitis and toxic bronchitis.

It is also preferable to use the medicament formulations according to the invention for preparing a pharmaceutical composition for the treatment of bronchiectasis.

It is also preferable to use the medicament formulations according to the invention for preparing a pharmaceutical composition for the treatment of ARDS (adult respiratory distress syndrome).

It is also preferable to use the medicament formulations according to the invention for preparing a pharmaceutical composition for the treatment of pulmonary oedema, for example toxic pulmonary oedema after aspiration or inhalation of toxic substances and foreign substances.

Most preferably, the present invention relates to the use of the pharmaceutical formulations according to the invention for preparing a pharmaceutical composition for the treatment of asthma or COPD. Also of particular importance is the above-mentioned use for preparing a pharmaceutical composition for once-a-day treatment of inflammatory and obstructive respiratory complaints, particularly for the once-a-day treatment of asthma or COPD.

Moreover the present invention relates to a process for the treatment of the above-mentioned diseases, characterised in that one or more of the above-mentioned medicament formulations according to the invention are administered in therapeutically effective amounts.

The present invention relates to liquid active substance formulations of these compounds which can be administered by inhalation; the liquid formulations according to the invention have to meet high quality standards. The formulations according to the invention may be inhaled by oral or nasal route. To achieve an optimum distribution of the active substances in the lung it makes sense to use a liquid formulation without propellant gases administered using suitable inhalers. A formulation of this kind may be inhaled both by oral route and by nasal route. Those inhalers which are capable of nebulising a small amount of a liquid formulation in the dosage needed for therapeutic purposes within a few seconds into an aerosol suitable for therapeutic inhalation are particularly suitable. Within the scope of the invention, preferred nebulisers are those in which an amount of less than 100 microlitres, preferably less than 50 microlitres, most preferably less than 25 microlitres of active substance solution can be nebulised preferably in one puff or two puffs to form an aerosol having an average particle size of less than 20 microns, preferably less than 10 microns, so that the inhalable part of the aerosol already corresponds to the therapeutically effective quantity.

An apparatus of this kind for the propellant-free administration of a metered amount of a liquid pharmaceutical composition for inhalation is described in detail for example in International Patent Application WO 91/14468 “Atomizing Device and Methods” and also in WO 97/12687, cf. FIGS. 6a and 6b and the accompanying description. In a nebuliser of this kind a pharmaceutical solution is converted by means of a high pressure of up to 500 bar into an aerosol destined for the lungs, which is sprayed. Within the scope of the present specification reference is expressly made to the entire contents of the literature mentioned above.

In inhalers of this kind the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulise the solution a special nozzle is used as described for example in WO 94/07607 or WO 99/16530. Reference is expressly made here to both these publications.

The aim of the invention is to provide an aqueous, ethanolic or aqueous-ethanolic formulation of the compound of formula 1 which meets the high standards required to ensure optimum nebulisation of a solution using the inhalers mentioned above. The active substance formulations according to the invention must be of sufficiently high pharmaceutical quality, i.e. they should be pharmaceutically stable over a storage time of some years, preferably at least one year, more preferably two years. These propellant-free formulations of solutions must also be capable of being nebulised by means of an inhaler under pressure, while the composition delivered in the aerosol produced is within a specified range.

Within the scope of the present invention, those compounds of formula 1 are preferably used wherein X⁻ is selected from among the chloride, maleate, salicylate, fumarate or succinate, optionally in the form of the hydrates and solvates thereof.

Particularly preferred, within the scope of the present invention, are the formulations which contain the compound of formula 1 wherein X⁻ denotes chloride.

References to the compound of formula 1 always include within the scope of the present invention all possible amorphous and crystalline modifications of this compound. References to the compound of formula 1 also include within the scope of the present invention all the possible solvates and hydrates which may be formed from this compound.

Any reference to the compound 1′ within the scope of the present invention is to be regarded as a reference to the pharmacologically active free base of the following formula

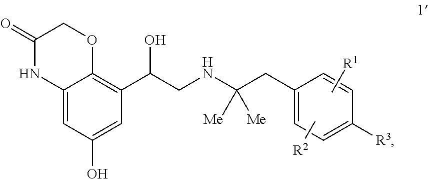

contained in the salts 1 wherein the groups R¹, R², R³ and X⁻ may have the meanings given above.

In another aspect the present invention relates to medicament formulations containing as the sole active substance a free base of formula 1′ wherein the groups R¹, R², R³ and X⁻ may have the meanings given above, optionally in the form of their tautomers, enantiomers, mixtures of enantiomers, racemates or solvates, at least one pharmacologically acceptable acid, optionally other pharmacologically acceptable excipients and/or complexing agents and, as solvent, water, ethanol or a mixture of water and ethanol.

According to the invention the formulation preferably contains only one compound of formula 1. However, the formulation may also contain a mixture of different salts of formula 1. If the medicament formulations according to the invention contain different salts of formula 1 the preferred formulations according to the invention are those wherein the various salts denote different salts of the same free base of formula 1′. Formulations which contain active substances other than those of formula 1 are not included in the invention.

The concentration of the compound of formula 1 based on the proportion of pharmacologically active free base 1′ in the pharmaceutical preparation according to the invention is about 0.1 to 1600 mg per 100 ml, according to the invention, preferably about 0.5 to 1000 mg per 100 ml, particularly preferably 0.75 to 200 mg per 100 ml. Particularly preferably, 100 ml of the formulations according to the invention contain about 1 to about 100 mg of 1′.

The pH of the formulation according to the invention is preferably between 2.0 and 6.5, preferably between 2.2 and 5.0, more preferably between about 3.0 and 4.5.

The pH is adjusted by the addition of pharmacologically acceptable acids. Pharmacologically acceptable inorganic acids or organic acids may be used for this purpose. Examples of preferred inorganic acids are selected from the group consisting of hydrochloric acid, hydrobromic acid, nitric acid, sulphuric acid and phosphoric acid. Examples of particularly suitable organic acids are selected from the group consisting of ascorbic acid, citric acid, malic acid, tartaric acid, maleic acid, succinic acid, fumaric acid, acetic acid, formic acid and propionic acid. Preferred inorganic acids are hydrochloric acid and sulphuric acid, of which hydrochloric acid is particularly preferred according to the invention. Of the organic acids, ascorbic acid, fumaric acid and citric acid are preferred. If desired, mixtures of the abovementioned acids may also be used, particularly in the case of acids which have other properties in addition to their acidifying properties, e.g. those which act as flavourings or antioxidants, such as for example citric acid or ascorbic acid.

If desired, pharmacologically acceptable bases may be used to titrate the pH precisely. Suitable bases include for example alkali metal hydroxides and alkali metal carbonates. The preferred alkali metal ion is sodium. If bases of this kind are used, care must be taken to ensure that the resulting salts, which are then contained in the finished pharmaceutical formulation, are pharmacologically compatible with the abovementioned acid.

The formulations according to the invention may contain complexing agents as other ingredients. By complexing agents are meant within the scope of the present invention molecules which are capable of entering into complex bonds. Preferably, these compounds should have the effect of complexing cations, most preferably metal cations. The formulations according to the invention preferably contain editic acid (EDTA) or one of the known salts thereof, e.g. sodium EDTA or disodium EDTA dihydrate (sodium edetate), as complexing agent. Preferably, sodium edetate is used, optionally in the form of its hydrates, more preferably in the form of its dihydrate. If complexing agents are used within the formulations according to the invention, their content is preferably in the range from 1 to 50 mg per 100 ml, more preferably in the range from 2 to 15 mg per 100 ml of the formulation according to the invention. Preferably, the formulations according to the invention contain a complexing agent in an amount of about 4 to 12 mg per 100 ml, more preferably about 10 mg per 100 ml of the formulation according to the invention.

The remarks made concerning sodium edetate also apply analogously to other possible additives which are comparable to EDTA or the salts thereof, which have complexing properties and can be used instead of them, such as for example nitrilotriacetic acid and the salts thereof.

Other pharmacologically acceptable excipients may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives which prolong the shelf life of the finished pharmaceutical formulation, as well as flavourings, vitamins and/or other additives known in the art. The additives also include pharmacologically acceptable salts such as sodium chloride, for example.

The preferred excipients include antioxidants such as ascorbic acid, for example, provided that it has not already been used to adjust the pH, vitamin A, vitamin E, tocopherols and similar vitamins or provitamins occurring in the human body.

Preservatives can be added to protect the formulation from contamination with pathogenic bacteria. Suitable preservatives are those known from the prior art, particularly benzalkonium chloride or benzoic acid or benzoates such as sodium benzoate in the concentration known from the prior art. Preferably, benzalkonium chloride is added to the formulation according to the invention. The amount of benzalkonium chloride is between 1 mg and 50 mg per 100 ml of formulation, preferably about 2 to 15 mg per 100 ml, more preferably about 3 to 12 mg per 100 ml of the formulation according to the invention, most preferably about 4 to 10 mg per 100 ml of the formulation according to the invention. Benzalkonium chloride may also be used according to the invention in admixture with other preservatives.

Preferred formulations contain only benzalkonium chloride, sodium edetate and the acid needed to adjust the pH, in addition to the solvent water and the compounds of formula 1. The pharmaceutical formulations according to the invention containing compounds of formula 1 are preferably used in an inhaler of the kind described hereinbefore in order to produce the propellant-free aerosols according to the invention. At this point we should once again expressly mention the patent documents described hereinbefore, to which reference is hereby made.

As described at the beginning, a further developed embodiment of the preferred inhaler is disclosed in WO 97/12687 (cf. in particular FIGS. 6a and 6b and the associated passages of description). This nebuliser (RESPIMAT®) can advantageously be used to produce the inhalable aerosols according to the invention containing a tiotropium salt as active substance.

Because of its cylindrical shape and handy size of less than 9 to 15 cm long and 2 to 4 cm wide, the device can be carried anywhere by the patient. The nebuliser sprays a defined volume of the pharmaceutical formulation out through small nozzles at high pressures, so as to produce inhalable aerosols.

The preferred atomiser essentially consists of an upper housing part, a pump housing, a nozzle, a locking clamp, a spring housing, a spring and a storage container, characterised by
a pump housing fixed in the upper housing part and carrying at one end a nozzle body with the nozzle or nozzle arrangement,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing,
a lower housing part which is fitted onto the spring housing in the axial direction.

The hollow piston with valve body corresponds to a device disclosed in WO 97/12687. It projects partially into the cylinder of the pump housing and is disposed to be axially movable in the cylinder. Reference is made particularly to FIGS. 1-4—especially FIG. 3—and the associated passages of description in the abovementioned International Patent Application. At the moment of release of the spring the hollow piston with valve body exerts, at its high pressure end, a pressure of 5 to 60 Mpa (about 50 to 600 bar), preferably 10 to 60 Mpa (about 100 to 600 bar) on the fluid, the measured amount of active substance solution. Volumes of 10 to 50 microlitres are preferred, volumes of 10 to 20 microlitres are more preferable, whilst a volume of 10 to 15 microlitres per actuation is particularly preferred.

The valve body is preferably mounted at the end of the hollow piston which faces the nozzle body.

The nozzle in the nozzle body is preferably microstructured, i.e. produced by micro-engineering. Microstructured nozzle bodies are disclosed for example in WO-99/16530; reference is hereby made to the contents of this specification, especially FIG. 1 and the associated description.

The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns.

If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20 degrees to 160 degrees, preferably at an angle of 60 to 150 degrees, most preferably 80 to 100°.

The nozzle openings are preferably arranged at a spacing of 10 to 200 microns, more preferably at a spacing of 10 to 100 microns, still more preferably 30 to 70 microns. A spacing of 50 microns is most preferred.

The directions of spraying therefore meet in the region of the nozzle openings.

As already mentioned, the liquid pharmaceutical preparation hits the nozzle body at an entry pressure of up to 600 bar, preferably 200 to 300 bar and is atomised through the nozzle openings into an inhalable aerosol. The preferred particle sizes of the aerosol are up to 20 microns, preferably 3 to 10 microns.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. a helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists for example of a ring of plastics or metal which is inherently radially elastically deformable. The ring is arranged in a plane perpendicular to the axis of the atomiser. After the locking of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomiser, and the deformable ring is thereby deformed in the annular plane. Details of the construction of the locking clamping mechanism are described in WO 97/20590.

The lower housing part is pushed axially over the spring housing and covers the bearing, the drive for the spindle and the storage container for the fluid.

When the atomiser is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360 degrees, e.g. 180 degrees. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomised can be inserted in the atomiser one after another and then used. The storage container contains the aqueous aerosol preparation according to the invention.

The atomising process is initiated by gently pressing the actuating button. The clamping mechanism then opens the way for the power take-off component. The biased spring pushes the piston into the cylinder in the pump housing. The fluid emerges from the nozzle of the atomiser in the form of a spray.

Further details of the construction are disclosed in PCT applications WO 97/12683 and WO 97/20590, to which reference is hereby made.

The components of the atomiser (nebuliser) are made of a material suitable for their function. The housing of the atomiser and—if the function allows—other parts as well are preferably made of plastics, e.g. by injection moulding. For medical applications, physiologically acceptable materials are used.

FIGS. 6a/b of WO 97/12687 show the RESPIMAT® nebuliser with which the aqueous aerosol preparations according to the invention can advantageously be inhaled. FIG. 6a shows a longitudinal section through the atomiser with the spring under tension, FIG. 6b shows a longitudinal section through the atomiser with the spring released.

The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66).

The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebuliser described above is suitable for nebulising the aerosol preparations according to the invention to form an aerosol suitable for inhalation.

If the formulation according to the invention is nebulised using the method described above (RESPIMAT®), the mass expelled, in at least 97%, preferably at least 98% of all the actuations of the inhaler (puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

However, the formulation according to the invention can also be nebulised using inhalers other than those described above, for example jet-stream inhalers.

The present invention also relates to an inhalation kit consisting of one of the pharmaceutical preparations according to the invention described above and an inhaler suitable for nebulising this pharmaceutical preparation. The present invention preferably relates to an inhalation kit consisting of one of the pharmaceutical preparations according to the invention described above and the RESPIMAT® inhaler described above.

The examples of formulations given below serve as illustrations without restricting the subject matter of the present invention to the compositions shown by way of example.

### I. Preparation of the Compounds of Formula 1

### Example 1

### 6-hydroxy-8-{1-hydroxy-2-[2-(4-hydroxy-2,6-dimethyl-phenyl)-1,1-dimethyl-ethylamino]-ethyl}-4H-benzo[1,4]oxazin-3-one-methanesulphonate

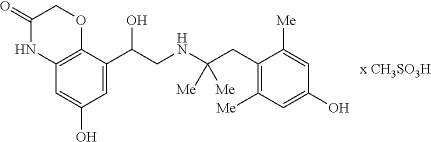

The compound is known from U.S. Pat. No. 4,460,581.

The examples of synthesis described below serve to illustrate new compounds according to the invention in more detail. However, they are intended only as examples of procedures to illustrate the invention without restricting it to the subject matter described in an exemplifying capacity hereinafter.

### Example 2

### 8-{2-[2-(4-fluoro-phenyl)-1,1-dimethyl-ethylamino]-1-hydroxy-ethyl}-6-hydroxy-4H-benzo[1,4]oxazin-3-one acid addition salt

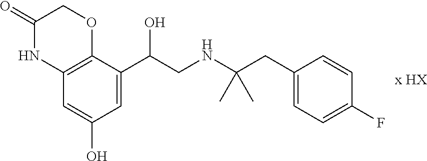

300 mg (0.91 mmol) 6-benzyloxy-8-(2,2-dihydroxy-acetyl)-4H-benzo[1,4]oxazin-3-one and 183 mg (1.09 mmol) 2-(4-fluoro-phenyl)-1,1-dimethyl-ethylamine were dissolved in 3 ml of ethanol. Molecular sieve was added and the mixture was heated for 30 minutes to 80° C. After cooling to ambient temperature 35 mg (0.91 mmol) sodium borohydride were added. The mixture was stirred for 1 hour at ambient temperature, sodium hydrogen carbonate solution was then added to the reaction mixture and it was extracted with ethyl acetate. The organic phases were evaporated down and the residue was chromatographed (eluant: hexane/ethyl acetate/methanol). The ethanolamine thus obtained (223 mg) was dissolved in methanol in order to cleave the benzyl protecting group and hydrogenated with 150 mg palladium hydroxide as catalyst at ambient temperature and normal pressure. The catalyst was separated off by filtration through Celite®, the filtrate was freed from the solvent and the residue was chromatographed (silica gel; eluant: dichloromethane/methanol). Beige solid. Yield: 76 mg (22%); mass spectrometry: [M+H]⁺=375. The product may be converted into the desired acid addition salt by reaction with the corresponding acid HX.

The (R)- and (S)-enantiomers of this embodiment may be obtained by separation of the racemate analogously to current methods known in the art.

### Example 3

### 6-hydroxy-8-{1-hydroxy-2-[2-(4-methoxy-phenyl)-1,1-dimethyl-ethylamino]-ethyl}-4H-benzo[1,4]oxazin-3-one hydrochloride

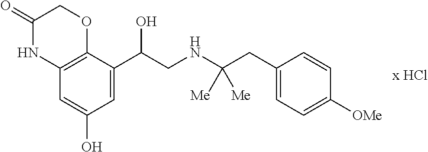

### a) 8-{2-[1,1-dimethyl-2-(4-methoxy-phenyl)-ethylamino]-1-hydroxy-ethyl}-6-benzyloxy-4H-benzo[1,4]oxazin-3-one

7.5 g (6-benzyloxy-4H-benzo[1,4]oxazin-3-one)-glyoxalhydrate are added at 70° C. to a solution of 3.6 g 1,1-dimethyl-2-(4-methoxyphenyl)-ethylamine in 100 mL ethanol and the mixture is stirred for 15 minutes. Then within 30 minutes at 10 to 20° C. 1 g sodium borohydride is added. The mixture is stirred for one hour, combined with 10 mL acetone and stirred for a further 30 minutes. The reaction mixture is diluted with 150 mL ethyl acetate, washed with water, dried with sodium sulphate and evaporated down. The residue is dissolved in 50 mL methanol and 100 mL ethyl acetate and acidified with conc. hydrochloric acid. After the addition of 100 mL diethyl ether the product is precipitated out. The crystals are filtered off, washed and recrystallised from 50 mL ethanol.

Yield: 7 g (68%; hydrochloride); m.p.=232-234° C.

### b) 8-{2-[1,1-dimethyl-2-(4-methoxy-phenyl)-ethylamino]-1-hydroxy-ethyl}-6-hydroxy-4H-benzo[1,4]oxazin-3-one-hydrochloride

6.8 g of the benzyl compound obtained previously are hydrogenated in 125 mL methanol with the addition of 1 g palladium on charcoal (5%) at ambient temperature and normal pressure. The catalyst is filtered off and the filtrate is freed from the solvent. After recrystallisation of the residue from 50 mL acetone and a little water a solid is obtained, which is filtered off and washed.

Yield: 5.0 g (89%; hydrochloride); m.p.=155-160° C.

The (R)- and (S)-enantiomers of Example 3 may be obtained from the racemate for example by means of chiral HPLC (e.g. column: Chirobiotic T, 250×22.1 mm obtained from Messrs Astec). Methanol with 0.05% triethylamine and 0.05% acetic acid may be used as the mobile phase. Silica gel with a particle size of 5 μm, to which the glycoprotein teicoplanin is covalently bound may be used as the column material. Retention time (R-enantiomer)=40.1 min, retention time (S-enantiomer)=45.9 min. The two enantiomers are obtained in the form of the free bases by this method and may be converted into the corresponding acid addition salts by reaction with the desired acid (e.g. hydrochloric acid) by the methods generally known in the prior art.

Of outstanding importance according to the invention is the R-enantiomer of Example 3.

### Example 4

### 6-hydroxy-8-{1-hydroxy-2-[2-(4-phenoxyethyl-acetate)-1,1-dimethyl-ethylamino]-ethyl}-4H-benzo[1,4]oxazin-3-one-hydrochloride

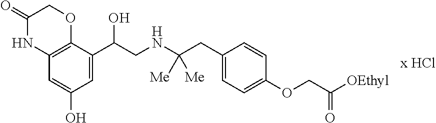

### a) 8-{2-[1,1-dimethyl-2-(4-phenoxyethyl-acetate)-ethylamino]-1-hydroxy-ethyl}-6-benzyloxy-4H-benzo[1,4]oxazin-3-one

Analogously to the method described in Example 3a) the title compound is obtained from 15 g (6-benzyloxy-4H-benzo[1,4]oxazin-3-one)-glyoxalhydrate and 11.8 g 1,1-dimethyl-2-(4-phenoxy-acetate ethyl)-ethylamine hydrochloride.

Yield: 16.5 g (69%, hydrochloride); m.p.=212-214° C.

### b) 8-{2-[1,1-dimethyl-2-(4-phenoxy-acetate ethyl)-ethylamino]-1-hydroxy-ethyl}-6-hydroxy-4H-benzo[1,4]oxazin-3-one-hydrochloride

8 g of the benzylalcohol obtained previously are dissolved in 100 mL ethanol, 100 mL methanol and 10 mL water and hydrogenated in the presence of 1 g palladium on charcoal (5%). After uptake of the theoretical amount of hydrogen calculated the catalyst is filtered off and the filtrate is evaporated down. The product that crystallises out when the solvent is distilled off is suction filtered and washed.

Yield: 5.5 g (81%; hydrochloride); m.p.=137-140° C.

The (R)- and (S)-enantiomers of this embodiment may be obtained by separation of the racemate analogously to current methods known in the art.

### Example 5

### 6-hydroxy-8-{1-hydroxy-2-[2-(4-phenoxy-acetic acid)-1,1-dimethyl-ethylamino]-ethyl}-4H-benzo[1,4]oxazin-3-one-hydrochloride

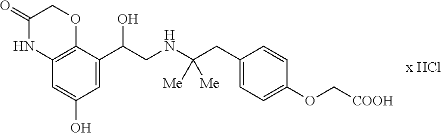

11 g 8-{2-[1,1-dimethyl-2-(4-phenoxyethyl-acetate)-ethylamino]-1-hydroxy-ethyl}-6-benzyloxy-4H-benzo[1,4]oxazin-3-one hydrochloride (Example 4a) are dissolved in 125 mL methanol and hydrogenated in the presence of 1 g palladium on charcoal (5%). After uptake of the theoretically calculated amount of hydrogen the catalyst is filtered off. 2.6 g sodium hydroxide dissolved in 20 mL water are added to the filtrate. The mixture is refluxed for 30 minutes, the methanol is distilled off and the remaining mixture is combined with 10 mL water, 20 mL n-butanol and 3.9 mL acetic acid. The precipitated solid is suction filtered and washed with diethyl ether.

Yield: 7 g (87%). The hydrochloride is obtained by recrystallisation from 0.5 molar hydrochloric acid. M.p.=152° C.

The (R)- and (S)-enantiomers of this embodiment may be obtained by separation of the racemate analogously to current methods known in the art.

### Example 6

### 8-{2-[1,1-dimethyl-2-(2,4,6-trimethylphenyl)-ethylamino]-1-hydroxy-ethyl}-6-hydroxy-4H-benzo[1,4]oxazin-3-one-hydrochloride

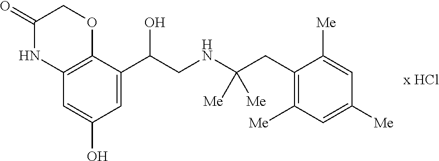

### a) 1-(6-benzyloxy-4H-benzo[1,4]oxazin-3-one)-2-[1,1-dimethyl-2-(2,4,6-trimethylphenyl)-ethylimino]-ethanone

7.2 g (6-benzyloxy-4H-benzo[1,4]oxazin-3-one)-glyoxalhydrate and 3.6 g 1,1-dimethyl-2-(2,4,6-trimethylphenyl)-ethylamine are heated to 70° C. for hour in 100 mL ethanol. After cooling the crystals precipitated are filtered off and washed with ethanol and diethyl ether.

Yield: 8.6 g (94%); m.p.=175° C.

### b) 8-{2-[1,1-dimethyl-2-(2,4,6-trimethylphenyl)-ethylamino]-1-hydroxy-ethyl}-6-benzyloxy-4H-benzo[1,4]oxazin-3-one

8.6 g of the Schiff-base obtained according to the prescribed method 6a) are dissolved in 100 mL ethanol and 20 mL THF, combined with 0.7 g sodium borohydride within 30 min at 10-20° C. and stirred for one hour. After the addition of 10 mL acetone the mixture is stirred for 30 minutes and then diluted with ethyl acetate and water. The product that crystallises out during acidification with conc. hydrochloric acid is filtered off and washed.

Yield: 7.4 g (80%, hydrochloride); m.p.=235° C. (decomposition).

### c) 8-{2-[1,1-dimethyl-2-(2,4,6-trimethylphenyl)-ethylamino]-1-hydroxy-ethyl}-6-hydroxy-4H-benzo[1,4]oxazin-3-one-hydrochloride

7.4 g of the benzyl compound obtained in Step b) are hydrogenated in 125 mL methanol with the addition of 1 g palladium on charcoal (5%) at ambient temperature and normal pressure. Then the catalyst is filtered off and the filtrate is evaporated down. The product that crystallises out on the addition of acetone is suction filtered and washed with acetone and diethyl ether. Yield: 5 g (78%, hydrochloride); m.p. 160° C. (decomposition).

The (R)- and (S)-enantiomers of this embodiment may be obtained by separation of the racemate analogously to current methods known in the art.

### Example 7

### 6-hydroxy-8-{1-hydroxy-2-[2-(4-hydroxy-phenyl)-1,1-dimethyl-ethylamino]-ethyl}-4H-benzo[1,4]oxazin-3-one-hydrochloride

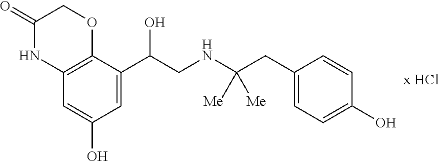

### a) 8-{2-[1,1-dimethyl-2-(4-hydroxy-phenyl)-ethylamino]-1-hydroxy-ethyl}-6-benzyloxy-4H-benzo[1,4]oxazin-3-one

The title compound is prepared from 10 g (6-benzyloxy-4H-benzo[1,4]oxazin-3-one)-glyoxalhydrate and 4.6 g 1,1-dimethyl-2-(4-hydroxy-phenyl)-ethylamine analogously to the procedure laid down for Example 3a).

Yield: 9.0 g (64%, hydrochloride); m.p.=255-258° C.

### b) 8-{2-[1,1-dimethyl-2-(4-hydroxy-phenyl)-ethylamino]-1-hydroxy-ethyl}-6-hydroxy-4H-benzo[1,4]oxazin-3-one-hydrochloride

5.7 g of the coupling product obtained previously are hydrogenated in the presence of 0.6 g palladium on charcoal (5%) in 100 mL methanol. After uptake of the theoretically calculated amount of hydrogen the catalyst is filtered off and the filtrate is freed from the solvent. The residue is dissolved in ethanol with heating and then combined with diethyl ether. The product precipitated is suction filtered and recrystallised once from water. Yield: 3.6 g (72%, hydrochloride); m.p.=159-162° C.

The (R)- and (S)-enantiomers of this embodiment may be obtained by separation of the racemate analogously to current methods known in the art.

### Example 8

### 6-hydroxy-8-{1-hydroxy-2-[2-(4-isopropyl-phenyl)-1,1dimethyl-ethylamino]-ethyl}-4H-benzo[1,4]oxazin-3-one-hydrochloride

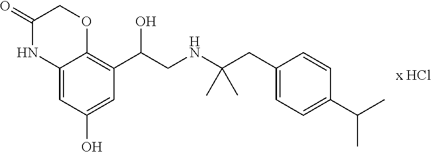

### a) 1-(4-isopropyl-phenyl)-2-methyl-propan-2-ol

The reaction of a Grignard compound, prepared from 20 g (119 mmol) 4-isopropylbenzyl chloride, with 11.4 ml (155 mmol) acetone yields the target compound as a colourless oil. Yield: 13.0 g (57%); mass spectrometry: [M+H]⁺=193.

### b) N-[2-(4-isopropyl-phenyl)-1,1-dimethyl-ethyl]-acetamide

A Ritter reaction is carried out with 10.2 g (53 mmol) 1-(4-isopropyl-phenyl)-2-methyl-propan-2-ol in the manner described for Example 9b). The reaction mixture is poured onto ice water and made alkaline with sodium hydroxide solution, during which time a solid is precipitated. This is suction filtered and dried.

Yield: 9.90 g (80%); mass spectrometry: [M+H]⁺=234.

### c) 2-(4-isopropyl-phenyl)-1,1-dimethyl-ethylamine

Reaction of 9.80 g (42 mmol) N-[2-(4-isopropyl-phenyl)-1,1-dimethyl-ethyl]-acetamide analogously to the procedure laid down for Example 9c).

Yield: 7.00 g (71%, hydrochloride); m.p. 202-206° C.

### d) 6-benzyloxy-8-{1-hydroxy-2-[2-(4-isopropyl-phenyl)-1,1-dimethyl-ethylamino]-ethyl}-4H-benzo[1,4]oxazin-3-one

2.18 g (6.1 mmol) benzyloxy-8-(2-ethoxy-2-hydroxy-acetyl)-4H-benzo[1,4]oxazin-3-one and 1.1 g (5.8 mmol) 2-(4-isopropyl-phenyl)-1,1-dimethyl-ethylamine are stirred for one hour at 50-80° C. in 40 mL ethanol. After cooling to ambient temperature 0.24 g (6.3 mmol) sodium borohydride are added. The mixture is stirred for one hour, diluted with 5 mL acetone and stirred for a further 30 minutes. The reaction mixture is acidified with hydrochloric acid, combined with 100 mL water and 80 mL ethyl acetate and made alkaline with ammonia. The organic phase is separated off, dried with sodium sulphate and freed from the solvent. The residue is dissolved in 20 mL ethyl acetate and 10 mL water, acidified with conc. hydrochloric acid and diluted with diethyl ether. After the addition of a crystallisation aid the precipitated solid is suction filtered and washed. White solid. Yield: 1.7 g (52%, hydrochloride); m.p. 220-222° C.

### e) 6-hydroxy-8-{1-hydroxy-2-[2-(4-isopropyl-phenyl)-1,1dimethyl-ethylamino]-ethyl}-4H-benzo[1,4]oxazin-3-one-hydrochloride

1.6 g (3.0 mmol) 6-benzyloxy-8-{1-hydroxy-2-[2-(4-isopropyl-phenyl)-1,1-dimethyl-ethylamino]-ethyl}-4H-benzo[1,4]oxazin-3-one are dissolved in methanol and hydrogenated with palladium on charcoal as catalyst at normal pressure and ambient temperature. The catalyst is suction filtered, the solvent is distilled off and the residue is recrystallised from isopropanol. White solid.

Yield: 1.1 g (85%, hydrochloride); m.p. 248-250° C.; mass spectrometry: [M+H]⁺=399.

The (R)- and (S)-enantiomers of this embodiment may be obtained by separation of the racemate analogously to current methods known in the art.

### Example 9

### 8-{2-[2-(4-ethyl-phenyl)-1,1-dimethyl-ethylamino]-1-hydroxy-ethyl}-6-hydroxy-4H-benzo[1,4]oxazin-3-one-hydrochloride

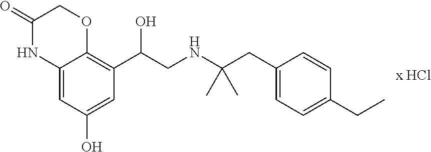

### a) 1-(4-ethyl-phenyl)-2-methyl-propan-2-ol

14.8 g (90 mmol) 1-(4-ethyl-phenyl)-propan-2-one, dissolved in diethyl ether, are added dropwise to 39 mL of a 3 molar solution of methylmagnesium bromide in diethyl ether, while cooling with the ice bath, in such a way that the temperature does not exceed 30° C. After the addition has ended the reaction mixture is refluxed for 1.5 hours and then hydrolysed with 10% ammonium chloride solution. After separation of the organic phase the aqueous phase is extracted with diethyl ether. The combined ether phases are washed with water, dried with sodium sulphate and evaporated down. The oil thus obtained is further reacted directly.

Yield: 15.5 g (90%).

### b) N-[2-(4-ethyl-phenyl)-1,1-dimethyl-ethyl]-acetamide

6.2 mL conc. sulphuric acid are added dropwise to 15.5 g (87 mmol) 1-(4-ethyl-phenyl)-2-methyl-propan-2-ol in 4.8 mL (91 mmol) acetonitrile and 15 mL glacial acetic acid within 15 minutes, during which time the temperature rises to 65° C. Then the mixture is stirred for one hour, diluted with ice water and made alkaline with conc. sodium hydroxide solution. After further stirring for 30 minutes the precipitated solid is suction filtered and washed with water. The crude product is dissolved in ethyl acetate, dried with sodium sulphate and evaporated down. The oil remaining is combined with petroleum ether, whereupon a solid is precipitated, which is filtered off and dried.

Yield: 16.3 g (85%); m.p. 90-92° C.

### c) 2-(4-ethyl-phenyl)-1,1-dimethyl-ethylamine

16.3 g (74 mmol) N-[2-(4-ethyl-phenyl)-1,1-dimethyl-ethyl]-acetamide and 8.0 g potassium hydroxide are refluxed for 15 hours in 60 mL ethyleneglycol. The reaction mixture is combined with ice water and extracted three times with diethyl ether. The combined organic phases are washed with water, dried with sodium sulphate and freed from the solvent. In order to prepare the hydrochloride the crude product is dissolved in acetonitrile and combined successively with ethereal hydrochloric acid and diethyl ether. The precipitated solid is suction filtered and dried.

Yield: 11.0 g (69%, hydrochloride); m.p. 165-167° C.

### d) 6-benzyloxy-8-{2-[2-(4-ethyl-phenyl)-1,1-dimethyl-ethyl amino]-1-hydroxy-ethyl}-4H-benzo[1,4]oxazin-3-one

The target compound is prepared analogously to the procedure laid down for Example 8d) from 2.14 g (6.0 mmol) 6-benzyloxy-8-(2-ethoxy-2-hydroxy-acetyl)-4H-benzo[1,4]oxazin-3-one and 1.0 g (5.6 mmol) 2-(4-ethyl-phenyl)-1,1-dimethyl-ethylamine. White solid. Yield: 1.7 g (54%, hydrochloride); m.p. 210-214° C.

### e) 8-{2-[2-(4-ethyl-phenyl)-1,1-dimethyl-ethylamino]-1-hydroxy-ethyl}-6-hydroxy-4H-benzo[1,4]oxazin-3-one-hydrochloride

The hydrogenolysis of 1.45 g (2.75 mmol) 6-benzyloxy-8-{2-[2-(4-ethyl-phenyl)-1,1-dimethyl-ethylamino]-1-hydroxy-ethyl}-4H-benzo[1,4]oxazin-3-one according to the prescribed method for Example 8e) yields the target compound in the form of a white solid.

Yield: 1.07 g (92%; hydrochloride); m.p. 266-269° C.; mass spectrometry: [M+H]⁺=385.

The (R)- and (S)-enantiomers of this embodiment may be obtained by separation of the racemate analogously to current methods known in the art.

### Example 10

### 8-{2-[2-(4-fluoro-3-methyl-phenyl)-1,1-dimethyl-ethylamino]-1-hydroxy-ethyl}-6-hydroxy-4H-benzo[1,4]oxazin-3-one-hydrochloride

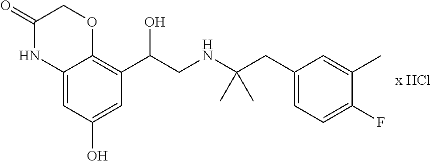

### a) 1-fluoro-2-methyl-4-(2-methyl-propenyl)-benzene

100 mL of a 0.5 molar solution of 4-fluoro-3-methyl-phenylmagnesium bromide in THF are combined within 30 minutes with 4.7 mL (50 mmol) isopropylaldehyde, during which time the temperature rises to 45° C. It is stirred for 30 minutes, refluxed for 1 hour and then hydrolysed with 10% ammonium chloride solution. After separation of the organic phase the mixture is extracted with diethyl ether. The organic phases are combined, dried and evaporated down. The alcohol thus obtained is dissolved in 100 mL toluene, combined with 1 g p-toluenesulphonic acid monohydrate and refluxed for three hours using the water separator. The reaction mixture is poured onto water and made alkaline with conc. sodium hydroxide solution. After separation of the organic phase the latter is washed with water, dried with sodium sulphate and freed from the solvent. Fractional distillation of the residue yields the product in the form of a colourless liquid (b.p. 80-85° C./10 mbar).

Yield: 4.1 g (50%).

### b) N-[2-(4-fluoro-3-methyl-phenyl)-1,1-dimethyl-ethyl]-formamide

4.9 mL conc. sulphuric acid are added dropwise at 5-15° C. to 1.5 g (31 mmol) sodium cyanide in 5 mL glacial acetic acid. The mixture is then combined with 3.9 g (24 mmol) 1-fluoro-2-methyl-4-(2-methyl-propenyl)-benzene, dissolved in 10 mL glacial acetic acid, and stirred for 1 hour at 50-60° C. The reaction mixture is diluted with ice water, made alkaline with conc. sodium hydroxide solution and extracted with dichloromethane. The organic phase is dried with sodium sulphate and freed from the solvent in vacuo. The light yellow oil thus obtained is further reacted directly. Yield: 4.3 g (87%).

### c) 2-(4-fluoro-3-methyl-phenyl)-1,1-dimethyl-ethylamine

4.3 g (20.6 mmol) N-[2-(4-fluoro-3-methyl-phenyl)-1,1-dimethyl-ethyl]-formamide, 20 mL conc. hydrochloric acid and 20 mL water are refluxed for 2 hours. The reaction mixture is diluted with water, made alkaline with conc. sodium hydroxide solution and extracted with dichloromethane. The organic phases are dried with sodium sulphate and evaporated down. The residue is dissolved in ethyl acetate, combined with ethereal hydrochloric acid and cooled. The precipitated crystals are suction filtered and washed with diethyl ether and dried. White solid.

Yield: 3.9 g (87%, hydrochloride); m.p. 196-198° C.

### d) 6-benzyloxy-8-{2-[2-(4-fluoro-3-methyl-phenyl)-1,1-dimethyl-ethylamino]-1-hydroxy-ethyl}-4H-benzo[1,4]oxazin-3-one

1.10 g (3.1 mmol) benzyloxy-8-(2-ethoxy-2-hydroxy-acetyl)-4H-benzo[1,4]oxazin-3-one and 0.50 g (2.8 mmol) 2-(4-fluoro-3-methyl-phenyl)-1,1-dimethyl-ethylamine are reacted and worked up analogously to the procedure laid down for Example 8d). White solid. Yield: 0.75 g (47%, hydrochloride); m.p. 228-230° C.

### e) 8-{2-[2-(4-fluoro-3-methyl-phenyl)-1,1-dimethyl-ethylamino]-1-hydroxy-ethyl}-6-hydroxy-4H-benzo[1,4]oxazin-3-one-hydrochloride

The hydrogenation of 0.70 g (1.4 mmol) 6-benzyloxy-8-{2-[2-(4-fluoro-3-methyl-phenyl)-1,1-dimethyl-ethylamino]-1-hydroxy-ethyl}-4H-benzo[1,4]oxazin-3-one yields the target compound as a white solid.

Yield: 0.50 g (87%, hydrochloride); m.p. 278-280° C.; mass spectroscopy: [M+H]⁺=389.

The (R)- and (S)-enantiomers of this embodiment may be obtained by separation of the racemate analogously to current methods known in the art.

### Example 11

### 8-{2-[2-(4-fluoro-2-methyl-phenyl)-1,1-dimethyl-ethylamino]-1-hydroxy-ethyl}-6-hydroxy-4H-benzo[1,4]oxazin-3-one-hydrochloride

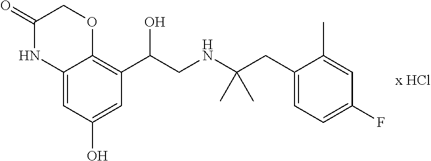

### a) 1-(4-fluoro-2-methyl-phenyl)-2-methyl-propyl acetate

500 mL of a 0.5 molar solution of 4-fluoro-6-methylphenylmagnesium bromide and 23.2 mL (260 mmol) isopropylaldehyde are reacted analogously to Example 10a). After hydrolysis with 10% ammonium chloride solution the aqueous phase is separated off and extracted with diethyl ether. The combined organic phases are dried with sodium sulphate and evaporated down. The alcohol thus obtained is then dissolved in 50 mL acetic anhydride, combined with 1 mL conc. sulphuric acid and stirred for three hours at reflux temperature. Then the reaction mixture is poured onto water, stirred for a further hour and made alkaline. It is extracted with dichloromethane, the organic phases are dried with sodium sulphate and the solvents are distilled off. Fractional distillation of the residue yields the product in the form of a colourless liquid (b.p. 105-110° C./8 mbar).

Yield 29.0 g (52%).

### b) N-[2-(4-fluoro-2-methyl-phenyl)-1,1-dimethyl-ethyl]-formamide

29.0 g (130 mmol) 1-(4-fluoro-2-methyl-phenyl)-2-methyl-propyl acetate are reacted and worked up analogously to the procedure laid down for Example 10b). Yellow oil. Yield: 27.0 g (99%).

### c) 2-(4-fluoro-2-methyl-phenyl)-1,1-dimethyl-ethylamine

In order to prepare the amine 27.0 g (130 mmol) N-[2-(4-fluoro-2-methyl-phenyl)-1,1-dimethyl-ethyl]-formamide are reacted as described in the procedure laid down for Example 10c). White solid. Yield: 15.5 g (55%, hydrochloride); m.p. 277-280° C.

### d) 6-benzyloxy-8-{2-[2-[4-fluoro-2-methyl-phenyl)-1,1-dimethyl-ethylamino]-1-hydroxy-ethyl}-4H-benzo[1,4]oxazin-3-one

Prepared analogously to the procedure laid down for Example 8d) from 0.95 g (2.66 mmol) benzyloxy-8-(2-ethoxy-2-hydroxy-acetyl)-4H-benzo[1,4]oxazin-3-one and 0.43 g (2.37 mmol) 2-(4-fluoro-2-methyl-phenyl)-1,1-dimethyl-ethylamine.

Yield: 0.75 g (55%, hydrochloride); m.p. 233-236° C.

### e) 8-{2-[2-(4-fluoro-2-methyl-phenyl)-1,1-dimethyl-ethylamino]-1-hydroxy-ethyl}-6-hydroxy-4H-benzo[1,4]oxazin-3-one-hydrochloride

The debenzylation of 0.70 g (1.36 mmol) 6-benzyloxy-8-{2-[2-[4-fluoro-2-methyl-phenyl)-1,1-dimethyl-ethylamino]-1-hydroxy-ethyl}-4H-benzo[1,4]oxazin-3-one yields the target compound in the form of a white solid.

Yield: 0.50 g (87%, hydrochloride); m.p. 278-280° C.; mass spectroscopy: [M+H]⁺=389.

The (R)- and (S)-enantiomers of this embodiment may be obtained by separation of the racemate analogously to current methods known in the art.

### Example 12

### 8-{2-[2-(2,4-difluoro-phenyl)-1,1-dimethyl-ethylamino]-1-hydroxy-ethyl}-6-hydroxy-4H-benzo[1,4]oxazin-3-one-hydrochloride

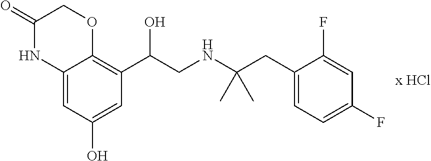

### a) 1-(2,4-difluoro-phenyl)-2-methyl-propan-2-ol

11.0 mL acetone, diluted with 50 mL diethyl ether, are added dropwise within 20 minutes to a solution of 500 mL of 0.25 molar 2,4-difluorobenzylmagnesium bromide in diethyl ether. Then the mixture is stirred for 1.5 hours at reflux temperature and then hydrolysed with 10% ammonium chloride solution. The ether phase is separated off, washed with water, dried with sodium sulphate and evaporated down. The fractional distillation of the residue yields the alcohol as a colourless liquid (b.p. 70-73° C./2 mmbar).

Yield: 20.0 g (86%).

### b) N-[2-(2,4-difluoro-phenyl]-1,1-dimethyl-ethyl]-formamide

Ritter reaction with 20 g (110 mmol) 1-(2,4-difluoro-phenyl)-2-methyl-propan-2-ol according to the process described for Example 10b). Yellow oil. Yield: 22.0 g (94%).

### c) 2-(2,4-difluoro-phenyl)-1,1-dimethyl-ethylamine

Reaction of 22.0 g (100 mmol) N-[2-(2,4-difluoro-phenyl]-1,1-dimethyl-ethyl]-formamide analogously to the procedure laid down for Example 10c).

Yield: 16.0 g (72%, hydrochloride); m.p. 201-203° C.

### d) 6-benzyloxy-8-{2-[2-(2,4-difluoro-phenyl)-1,1-dimethyl-ethylamino]-1-hydroxy-ethyl}-4H-benzo[1,4]oxazin-3-one

Reaction of 0.89 g (2.49 mmol) benzyloxy-8-(2-ethoxy-2-hydroxy-acetyl)-4H-benzo[1,4]oxazin-3-one and 0.40 g (2.16 mmol) 2-(2,4-difluoro-phenyl)-1,1-dimethyl-ethylamine in the manner described for Example 8d).

Yield: 0.80 g (62%, hydrochloride); m.p. 245-247° C.

### e) 8-{2-[2-(2,4-difluoro-phenyl)-1,1-dimethyl-ethylamino]-1-hydroxy-ethyl}-6-hydroxy-4H-benzo[1,4]oxazin-3-one-hydrochloride

The hydrogenolysis of 0.70 g (1.35 mmol) 6-benzyloxy-8-{2-[2-(2,4-difluoro-phenyl)-1,1-dimethyl-ethylamino]-1-hydroxy-ethyl}-4H-benzo[1,4]oxazin-3-one yields the target compound as a white solid.

Yield: 0.48 g (83%, hydrochloride); m.p. 279-280° C.; mass spectroscopy: [M+H]⁺=393.

The (R)- and (S)-enantiomers of this embodiment may be obtained by separation of the racemate analogously to current methods known in the art.

### Example 13

### 8-{2-[2-(3,5-difluoro-phenyl)-1,1-dimethyl-ethylamino]-1-hydroxy-ethyl}-6-hydroxy-4H-benzo[1,4]oxazin-3-one-hydrochloride

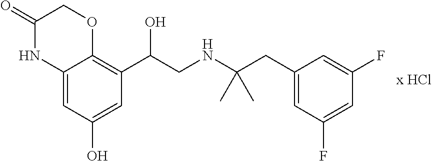

### a) 1-(3,5-difluoro-phenyl)-2-methyl-propan-2-ol

The target compound is obtained by reacting a Grignard compound, prepared from 25.0 g (121 mmol) 3,5-difluorobenzylbromide, with 12.6 mL (171 mmol) acetone. Yellow oil.

Yield: 13.5 g (60%).

### b) 2-(3,5-difluoro-phenyl)-1,1-dimethyl-ethylamine

The Ritter reaction of 5.5 g (29.5 mmol) 1-(3,5-difluoro-phenyl)-2-methyl-propan-2-ol and 1.8 g sodium cyanide yields 7.0 g formamide, which is treated with hydrochloric acid in order to cleave the formyl group. Light yellow oil. Yield: 4.6 g (75%).

### c) 6-benzyloxy-8-{2-[2-(3,5-difluoro-phenyl)-1,1-dimethyl-ethylamino]-1-hydroxy-ethyl}-4H-benzo[1,4]oxazin-3-one

Prepared from 1.73 g (4.84 mmol) benzyloxy-8-(2-ethoxy-2-hydroxy-acetyl)-4H-benzo[1,4]oxazin-3-one and 0.80 g (4.32 mmol) 2-(3,5-difluoro-phenyl)-1,1-dimethyl-ethylamine in the usual way. Yield: 1.50 g (58%, hydrochloride); m.p. 240-244° C.

### d) 8-{2-[2-(3,5-difluoro-phenyl)-1,1-dimethyl-ethylamino]-1-hydroxy-ethyl}-6-hydroxy-4H-benzo[1,4]oxazin-3-one-hydrochloride

Hydrogenolysis of 1.30 g (2.43 mmol) 6-benzyloxy-8-{2-[2-(3,5-difluoro-phenyl)-1,1-dimethyl-ethylamino]-1-hydroxy-ethyl}-4H-benzo[1,4]oxazin-3-one yields the target compound as a white solid.

Yield: 0.90 g (86%, hydrochloride); m.p. 150-158° C.; mass spectroscopy: [M+H]⁺=393.

The (R)- and (S)-enantiomers of this embodiment may be obtained by separation of the racemate analogously to current methods known in the art.

### Example 14

### 8-{2-[2-(4-ethoxy-phenyl)-1,1-dimethyl-ethylamino]-1-hydroxy-ethyl}-6-hydroxy-4H-benzo[1,4]oxazin-3-one-hydrochloride

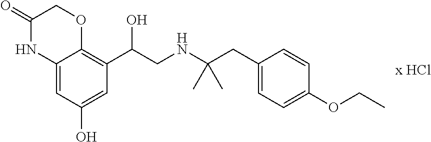

### a) benzyl[2-(4-ethoxy-phenyl)-1,1-dimethyl-ethyl]-carbamate

15.0 g (50 mmol) benzyl [2-(4-hydroxy-phenyl)-1,1-dimethyl-ethyl]-carbamate are stirred with 7.5 mL (92 mmol) ethyl iodide and 21 g (150 mmol) potassium carbonate for 10 hours at 90-100° C. The reaction mixture is combined with ethyl acetate, washed twice with water and dried with sodium sulphate. After removal of the solvents by distillation a yellow oil remains (15.0 g, 92%), which is further reacted directly.

### b) 2-(4-ethoxy-phenyl)-1,1-dimethyl-ethylamine

A solution of 15.0 g (49 mmol) benzyl [2-(4-ethoxy-phenyl)-1,1-dimethyl-ethyl]-carbamate in 100 mL glacial acetic acid is combined with 2 g palladium on charcoal (10%) and then hydrogenated at 5 bar and 40 to 50° C. The catalyst is filtered off and the filtrate is freed from the solvent. The residue is dissolved in a little water, made alkaline with conc. sodium hydroxide solution and extracted with ethyl acetate. The organic phase is washed with water, dried with sodium sulphate and evaporated down. The crude product is dissolved in acetonitrile and acidified with ethereal hydrochloric acid. The solid precipitated after the addition of diethyl ether is suction filtered and dried.

Yield: 8.8 g (hydrochloride, 84%); m.p. 198-200° C.

### c) 6-benzyloxy-8-{2-[2-(4-ethoxy-phenyl)-1,1-dimethyl-ethylamino]-1-hydroxy-ethyl}-4H-benzo[1,4]oxazin-3-one

2.14 g (6.0 mmol) 6-benzyloxy-8-(2-ethoxy-2-hydroxy-acetyl)-4H-benzo[1,4]oxazin-3-one and 1.0 g (5.2 mmol) 2-(4-ethoxy-phenyl)-1,1-dimethyl-ethylamine are stirred in 40 mL ethanol for one hour at 50-80° C. After cooling to ambient temperature 0.23 g (6.0 mmol) sodium borohydride are added and the mixture is stirred for another hour. The reaction mixture is combined with 5 ml acetone, stirred for 30 minutes, acidified with glacial acetic acid and evaporated down. The residue is combined with water and ethyl acetate and made alkaline. The organic phase is separated off, washed with water, dried with sodium sulphate and freed from the solvent in vacuo. The residue is again dissolved in ethyl acetate and water, combined with conc. hydrochloric acid and diluted with diethyl ether. The precipitated solid is suction filtered and washed with diethyl ether. White solid.

Yield: 2.0 g (61%, hydrochloride); m.p. 214-216° C.

### d) 8-{2-[2-(4-ethoxy-phenyl)-1,1-dimethyl-ethylamino]-1-hydroxy-ethyl}-6-hydroxy-4H-benzo[1,4]oxazin-3-one-hydrochloride

1.5 g (2.8 mmol) 6-benzyloxy-8-{2-[2-(4-ethoxy-phenyl)-1,1-dimethyl-ethylamino]-1-hydroxy-ethyl}-4H-benzo[1,4]oxazin-3-one in 80 mL methanol are hydrogenated with 250 mg palladium on charcoal (10%) as catalyst at ambient temperature and normal pressure. The catalyst is suction filtered and the filtrate is evaporated down. The residue is dissolved in 5 mL ethanol by heating, inoculated and diluted with ethyl acetate. The precipitated solid is filtered off and washed. White solid.

Yield 1.0 g (83%, hydrochloride); m.p. 232-235° C.; mass spectrometry: [M+H]⁺=401.

The (R)- and (S)-enantiomers of this embodiment may be obtained by separation of the racemate analogously to current methods known in the art.

### Example 15

### 8-{2-[2-(3,5-dimethyl-phenyl)-1,1-dimethyl-ethylamino]-1-hydroxy-ethyl}-6-hydroxy-4H-benzo[1,4]oxazin-3-one-hydrochloride

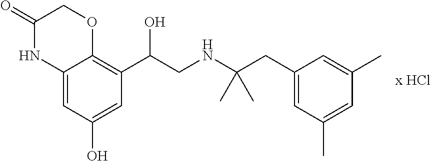

### a) 1-(3,5-dimethyl-phenyl)-2-methyl-propanol-2-ol

Obtained from the reaction of ethyl (3,5-dimethyl-phenyl)-acetate with methylmagnesium bromide.

### b) 2-(3,5-dimethyl-phenyl)-1,1-dimethyl-ethylamine

By reacting 6.00 g (34 mmol) 1-(3,5-dimethyl-phenyl)-2-methyl-propanol-2-ol and 2.00 g (41 mmol) sodium cyanide in a Ritter reaction, 2.40 g 2-(3,5-dimethyl-phenyl)-1,1-dimethyl-ethylformamide (35% yield) are obtained. To release the amine the formamide (2.40 g, 11.7 mmol) is treated with hydrochloric acid. The process and working up take place analogously to the procedure laid down for Example 10c). Oil.

Yield: 1.70 g (82%); mass spectroscopy: [M+H]⁺=178.

### c) 6-benzyloxy-8-{2-[2-(3,5-dimethyl-phenyl)-1,1-dimethyl-ethylamino]-1-hydroxy-ethyl}-4H-benzo[1,4]oxazin-3-one

Prepared analogously to the procedure laid down for Example 8d) from 1.47 g (4.1 mmol) benzyloxy-8-(2-ethoxy-2-hydroxy-acetyl)-4H-benzo[1,4]oxazin-3-one and 0.65 g (3.7 mmol) 2-(3,5-dimethyl-phenyl)-1,1-dimethyl-ethylamine.

Yield: 1.1 g (51%, hydrochloride); m.p. 220-222° C.

### d) 8-{2-[2-(3,5-dimethyl-phenyl)-1,1-dimethyl-ethylamino]-1-hydroxy-ethyl}-6-hydroxy-4H-benzo[1,4]oxazin-3-one-hydrochloride

The target compound was obtained after hydrogenolysis of 0.90 g (1.71 mmol) 6-benzyloxy-8-{2-[2-(3,5-dimethyl-phenyl)-1,1-dimethyl-ethylamino]-1-hydroxy-ethyl}-4H-benzo[1,4]oxazin-3-one and recrystallisation of the crude product from isopropanol. White solid.

Yield: 0.50 g (69%, hydrochloride); m.p. 235-238° C.; mass spectroscopy: [M+H]⁺=385.

The (R)- and (S)-enantiomers of this embodiment may be obtained by separation of the racemate analogously to current methods known in the art.

### Example 16

### Acid addition salt of 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid

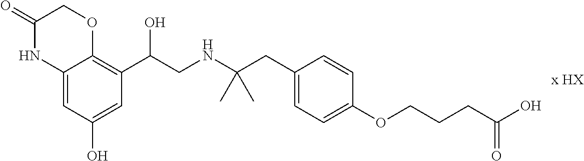

### a) ethyl 4-[4-(2-amino-2-methyl-propyl)-phenoxy]-butyrate

4.5 g (15.0 mmol) benzyl [2-(4-hydroxy-phenyl)-1,1-dimethyl-ethyl]-carbamate, 2.3 mL (16.0 mmol) ethyl 4-bromo-butyrate, 2.3 g (16.6 mmol) potassium carbonate and 0.3 g (1.8 mmol) potassium iodide in 20 mL dimethylformamide are heated for 13 hours at 120° C. The reaction mixture is diluted with ethyl acetate and washed successively with water, sodium hydroxide solution and water. The organic phase is dried with sodium sulphate and evaporated down. The residue is purified by chromatography (eluant: cyclohexane/ethyl acetate=9:1). 5.0 g of a yellow oil is isolated which is dissolved in 50 mL acetic acid and hydrogenated with 1.0 g palladium on charcoal as catalyst at 40° C. and 3 bar. The catalyst is filtered off and the filtrate is freed from solvent. The residue is dissolved in diethyl ether and combined with ethereal hydrochloric acid. The precipitated solid is suction filtered and dried.

Yield: 2.9 g (66% over two steps, hydrochloride); m.p.=103-105° C.

### b) ethyl 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyrate

1.20 g (3.36 mmol) benzyloxy-8-(2-ethoxy-2-hydroxy-acetyl)-4H-benzo[1,4]oxazin-3-one and 0.90 g (3.22 mmol) ethyl 4-[4-(2-amino-2-methyl-propyl)-phenoxy]-butyrate are reacted in the manner described for Example 8d). The crude product is dissolved in 10 mL ethyl acetate and 10 mL water and combined with oxalic acid with stirring. The solution is diluted with diethyl ether and the precipitated solid is suction filtered and washed with diethyl ether.

Yield: 1.20 g (54%, oxalate); m.p. 223-227° C.

### c) 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyric acid

A solution of 1.00 g (1.73 mmol) ethyl 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyrate in 25 mL methanol is combined with 2.5 mL 1N sodium hydroxide solution, refluxed for 30 minutes and then neutralised with 1 N hydrochloric acid. The solution is evaporated down and the oil remaining is dissolved by heating in 5 mL n-butanol. After the addition of a crystallisation aid a solid is precipitated, which is suction filtered and washed with acetone and diethyl ether. Yield: 0.75 g (79%); m.p. 216-218° C.

### d) 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid

0.70 g (1.28 mmol) 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyric acid are dissolved in 25 mL methanol and 2 mL acetic acid and hydrogenated in the presence of 150 mg palladium on charcoal (10%) at ambient temperature and normal pressure. The catalyst is filtered off and the filtrate is freed from the solvent. The product is obtained by crystallisation from a methanol/acetone mixture.

Yield: 0.40 g (68%); m.p. 201-204° C.; mass spectroscopy: [M+H]⁺=459.

The product may be converted into the desired acid addition salt by reaction with the corresponding acid HX.

The (R)- and (S)-enantiomers of this embodiment may be obtained by separation of the racemate analogously to current methods known in the art.

### Example 17

### 8-{2-[2-(3,4-difluoro-phenyl)-1,1-dimethyl-ethylamino]-1-hydroxy-ethyl}-6-hydroxy-4H-benzo[1,4]oxazin-3-one-trifluoroacetate

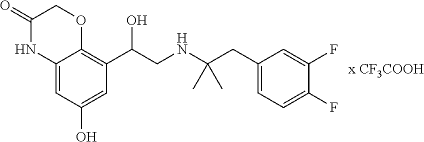

### a) 1-(3,4-difluoro-phenyl)-2-methyl-propan-2-ol

From 23.0 g (111 mmol) 3,4-difluorobenzylbromide a Grignard is prepared, which is then reacted with 11.6 mL (158 mmol) acetone. Light yellow oil.

Yield: 9.7 g (47%); R_{f} value: 0.55 (ethyl acetate/petroleum ether=1:3).

### b) N-[2-(3,4-difluoro-phenyl)-1,1-dimethyl-ethyl]-formamide

The target compound is obtained by Ritter reaction with 4.0 g (21.5 mmol) 1-(3,4-difluoro-phenyl)-2-methyl-propan-2-ol. Light yellow oil.

Yield: 4.0 g (87%); mass spectrometry: [M+H]⁺=214.

### c) 2-(3,4-difluoro-phenyl)-1,1-dimethyl-ethylamine

4.00 g (18.5 mmol) N-[2-(3,4-difluoro-phenyl)-1,1-dimethyl-ethyl]-formamide are dissolved in ethanol, combined with conc. hydrochloric acid and heated overnight at reflux temperature. The reaction solution is poured onto ice water, made alkaline with sodium hydroxide and extracted with tert-butylmethylether. The organic phases are washed with water, dried with sodium sulphate and evaporated down. Yellow oil.

Yield: 3.2 g (92%); mass spectrometry: [M+H]⁺=186.

### d) 8-{2-[2-(3,4-difluoro-phenyl)-1,1-dimethyl-ethylamino]-1-hydroxy-ethyl}-6-hydroxy-4H-benzo[1,4]oxazin-3-one

357 mg (1 mmol) 6-benzyloxy-8-(2-ethoxy-2-hydroxy-acetyl)-4H-benzo[1,4]oxazin-3-one and 185 mg (1 mmol) 2-(3,4-difluoro-phenyl)-1,1-dimethyl-ethylamine are stirred for 30 minutes in 5 mL tetrahydrofuran at ambient temperature. The mixture is cooled to 0° C. and under an argon atmosphere 1.5 mL of a 2 molar solution of lithium borohydride in tetrahydrofuran is added dropwise. The mixture is stirred for 30 min at ambient temperature, combined with 10 mL dichloromethane and 3 mL water, stirred for a further hour and then filtered through Extrelut®. The eluate containing the ethanolamine is freed from the solvent. The residue is dissolved in methanol and hydrogenated with palladium on charcoal (10%) as catalyst at 2.5 bar and ambient temperature. Then the catalyst is separated off and the crude product is purified by chromatography (acetonitrile/water/0,1% trifluoroacetic acid). White solid.

Yield: 31 mg (6%, trifluoroacetate); mass spectroscopy: [M+H]⁺=393.

The (R)- and (S)-enantiomers of this embodiment may be obtained by separation of the racemate analogously to current methods known in the art.

### Example 18

### 8-{2-[2-(2-chloro-4-fluoro-phenyl)-1,1-dimethyl-ethylamino]-1-hydroxy-ethyl}-6-hydroxy-4H-benzo[1,4]oxazin-3-one-trifluoroacetate

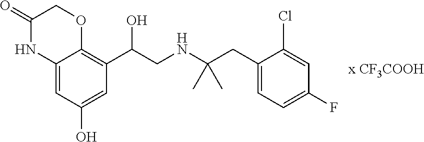

### a) 1-(2-chloro-4-fluoro-phenyl)-2-methyl-propan-2-ol

Prepared from 20 g (97 mmol) methyl (2-chloro-4-fluoro-phenyl)-acetate and 98 mL of a 3 molar solution of methylmagnesium bromide analogously to the procedure laid down for Example 8a).

### b) N-[2-(2-chloro-4-fluoro-phenyl)-1,1-dimethyl-ethyl]-formamide

7.5 g (37 mmol) 1-(2-chloro-4-fluoro-phenyl)-2-methyl-propan-2-ol were reacted and worked up according to the procedure described for Example 10b). The oil thus obtained was chromatographed for further purification on a short silica gel column (petroleum ether/ethyl acetate=9:1). Oil. Yield 7.4 g (87%); mass spectrometry: [M+H]⁺=230/232.

### c) 2-(2-chloro-4-fluoro-phenyl)-1,1-dimethyl-ethylamine

Reaction of 7.4 g (32 mmol) N-[2-(2-chloro-4-fluoro-phenyl)-1,1-dimethyl-ethyl]-formamide as described in the procedure laid down for Example 17c). Brown oil.

Yield: 5.14 g (79%); mass spectrometry: [M+H]⁺=202/204.

### d) 8-{2-[2-(2-chloro-4-fluoro-phenyl)-1,1-dimethyl-ethylamino]-1-hydroxy-ethyl}-6-hydroxy-4H-benzo[1,4]oxazin-3-one

357 mg (1 mmol) 6-benzyloxy-8-(2-ethoxy-2-hydroxy-acetyl)-4H-benzo[1,4]oxazin-3-one and 202 mg (1 mmol) 2-(2-chloro-4-fluoro-phenyl)-1,1-dimethyl-ethylamine are reacted analogously to the procedure laid down for Example 10d) with lithium borohydride. In order to debenzylate the ethanolamine thus obtained it is dissolved in 3 mL dichloromethane and cooled to −78° C. At this temperature 2 ml of a 1 molar solution of boron tribromide in dichloromethane is added dropwise and the mixture is allowed to come slowly up to ambient temperature. The reaction mixture is combined with 10 mL dichloromethane and 3 mL water and filtered through Extrelut®. The eluate is freed from the solvent and the residue is purified by chromatography (acetonitrile/water/0,1% trifluoroacetic acid). White solid.

Yield: 70 mg (13%, trifluoroacetate); mass spectroscopy: [M+H]⁺=409/11.

The (R)- and (S)-enantiomers of this embodiment may be obtained by separation of the racemate analogously to current methods known in the art.

### Example 19

### Acid addition salt of 8-{2-[2-(4-chloro-phenyl)-1,1-dimethyl-ethylamino]-1-hydroxy-ethyl}-6-hydroxy-4H-benzo[1,4]oxazin-3-one

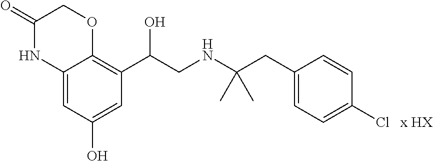

A solution of 300 mg (0.91 mmol) 6-benzyloxy-8-(2,2-dihydroxy-acetyl)-4H-benzo[1,4]oxazin-3-one and 200 mg (1.09 mmol) 2-(4-chloro-phenyl)-1,1-dimethyl-ethylamine in 3 mL ethanol was combined with molecular sieve and stirred for 90 minutes at 80° C. The mixture was left to cool to ambient temperature, 35 mg (0.91 mmol) sodium borohydride were added and the mixture was stirred for 1 hour. Then the reaction mixture was combined with sodium hydrogen carbonate solution and extracted with ethyl acetate. The combined organic phases were freed from the solvent and the residue was chromatographed (eluant: hexane/ethyl acetate/methanol), yielding 305 mg ethanolamine. This was dissolved in 3 mL dichloromethane and under an argon atmosphere cooled to −78° C. 3 mL of a 1 molar solution of boron tribromide in dichloromethane were added dropwise and the mixture was stirred for one hour at −78° C. and for 20 minutes at ambient temperature. Then at −78° C. 3 mL conc. ammonia solution were added dropwise and the mixture was stirred for 5 minutes. The reaction mixture was combined with ammonium chloride solution and extracted with ethyl acetate. The combined organic phases were evaporated down and the residue was chromatographed for further purification (silica gel; eluant: dichloromethane/methanol+1% ammonia). Beige solid: 93 mg (26%); mass spectrometry: [M+H]⁺=391. The product may be converted into the desired acid addition salt by reaction with the corresponding acid HX.

The (R)- and (S)-enantiomers of this embodiment may be obtained by separation of the racemate analogously to current methods known in the art.

### Example 20

### 8-{2-[2-(4-bromo-phenyl)-1,1-dimethyl-ethylamino]-1-hydroxy-ethyl}-6-hydroxy-4H-benzo[1,4]oxazin-3-one acid addition salt

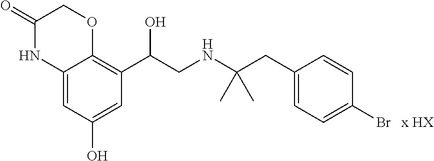

The preparation of the ethanolamine and debenzylation were carried out as described in Example 19 from 300 mg (0.91 mmol) 6-benzyloxy-8-(2,2-dihydroxy-acetyl)-4H-benzo[1,4]oxazin-3-one and 250 mg (1.09) mmol) 2-(4-bromo-phenyl)-1,1-dimethyl-ethylamine. Beige solid.

Yield: 54 mg (14%); mass spectrometry: [M+H]⁺=435, 437.

The product may be converted into the desired acid addition salt by reaction with the corresponding acid HX.

The (R)- and (S)-enantiomers of this embodiment may be obtained by separation of the racemate analogously to current methods known in the art.

The following compounds of formula 1 according to the invention may also be obtained analogously to the examples of synthesis described hereinbefore:

### Example 21

Acid addition salt of 8-{2-[2-(3-methyl-phenyl)-1,1-dimethyl-ethylamino]-1-hydroxy-ethyl}-6-hydroxy-4H-benzo[1,4]oxazin-3-one;

### Example 22

Acid addition salt of 8-{2-[2-(4-fluoro-3-methoxy-phenyl)-1,1-dimethyl-ethylamino]-1-hydroxy-ethyl}-6-hydroxy-4H-benzo[1,4]oxazin-3-one;

### Example 23

Acid addition salt of 8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;

### Example 24

Acid addition salt of 8-{2-[2-(4-chloro-2-methyl-phenyl)-1,1-dimethyl-ethylamino]-1-hydroxy-ethyl}-6-hydroxy-4H-benzo[1,4]oxazin-3-one;

### Example 25

Acid addition salt of 8-{2-[2-(4-chloro-3-fluoro-phenyl)-1,1-dimethyl-ethylamino]-1-hydroxy-ethyl}-6-hydroxy-4H-benzo[1,4]oxazin-3-one;

### Example 26

Acid addition salt of 8-{2-[2-(4-chloro-2-fluoro-phenyl)-1,1-dimethyl-ethylamino]-1-hydroxy-ethyl}-6-hydroxy-4H-benzo[1,4]oxazin-3-one;

### Example 27

Acid addition salt of 8-{2-[2-(3-chloro-4-fluoro-phenyl)-1,1-dimethyl-ethylamino]-1-hydroxy-ethyl}-6-hydroxy-4H-benzo[1,4]oxazin-3-one;

### Example 28

Acid addition salt of 8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;

### Example 29

Acid addition salt of 8-{2-[2-(2,5-difluoro-4-methoxy-phenyl)-1,1-dimethyl-ethylamino]-1-hydroxy-ethyl}-6-hydroxy-4H-benzo[1,4]oxazin-3-one;

### Example 30

Acid addition salt of 8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;

### Example 31

Acid addition salt of 8-{2-[2-(3,5-dichloro-phenyl)-1,1-dimethyl-ethylamino]-1-hydroxy-ethyl}-6-hydroxy-4H-benzo[1,4]oxazin-3-one;

### Example 32

Acid addition salt of 8-{2-[2-(4-chloro-3-methyl-phenyl)-1,1-dimethyl-ethylamino]-1-hydroxy-ethyl}-6-hydroxy-4H-benzo[1,4]oxazin-3-one;

### Example 33

Acid addition salt of 8-{2-[2-(3,4,5-trifluoro-phenyl)-1,1-dimethyl-ethylamino]-1-hydroxy-ethyl}-6-hydroxy-4H-benzo[1,4]oxazin-3-one;

### Example 34

Acid addition salt of -{2-[2-(3,4-dichloro-phenyl)-1,1-dimethyl-ethylamino]-1-hydroxy-ethyl}-6-hydroxy-4H-benzo[1,4]oxazin-3-one.

### II. Examples of Formulations

A) The Table that follows shows examples of formulations of the R-enantiomer of the compound of Example 1:

100 ml of pharmaceutical formulation contain in purified water or water for injections:

| | | benzalkonium | disodium edetate | citric |
|---|---|---|---|---|
| | 1 (1′-CH₃SO₃H) | chloride | dihydrate | acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 90 | 5 | 12 | 3 |
| 9 | 10 | 5 | 10 | 4 |
| 10 | 2.7 | 10 | 10 | 3 |
| 11 | 0.5 | 15 | 10 | 2 |

B) The Table that follows shows examples of formulations of the R-enantiomer of the compound of Example 3:

100 ml of pharmaceutical formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 90 | 10 | 10 | 3 |
| 9 | 23 | 10 | 10 | 3 |
| 10 | 2.7 | 10 | 10 | 3 |
| 11 | 0.5 | 15 | 10 | 2 |

C) The Table that follows shows examples of formulations of the R-enantiomer of the compound of Example 7:

100 ml of pharmaceutical formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 5 | 12 | 3 |
| 9 | 10 | 5 | 10 | 4 |
| 10 | 2.5 | 10 | 10 | 3 |
| 11 | 0.5 | 15 | 10 | 2 |

D) The Table that follows shows examples of formulations of the R-enantiomer of the compound of Example 9:

100 ml of pharmaceutical formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 5 | 12 | 3 |
| 9 | 10 | 5 | 10 | 4 |
| 10 | 2.5 | 10 | 10 | 3 |
| 11 | 0.5 | 15 | 10 | 2 |

E) The Table that follows shows examples of formulations of the R-enantiomer of the compound of Example 14:

100 ml of pharmaceutical formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 5 | 12 | 3 |
| 9 | 10 | 5 | 10 | 4 |
| 10 | 2.5 | 10 | 10 | 3 |
| 11 | 0.5 | 15 | 10 | 2 |

F) The Table that follows shows examples of formulations of the R-enantiomer of the compound of Example 17:

100 ml of pharmaceutical formulation contain in purified water or water for injections:

| | | benzalkonium | disodium edetate | citric |
|---|---|---|---|---|
| | 1 (1′-CF₃COOH) | chloride | dihydrate | acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 5 | 12 | 3 |
| 9 | 10 | 5 | 10 | 4 |
| 10 | 2.5 | 10 | 10 | 3 |
| 11 | 0.5 | 15 | 10 | 2 |

G) The Table that follows shows examples of formulations of the R-enantiomer of the compound of Example 3:

100 ml of pharmaceutical preparation contain:

| | | | disodium | | to 100 ml with |
|---|---|---|---|---|---|
| | 1 | benzalkonium | edetate | citric | ethanol/water |
| | (1′-HCl) | chloride | dihydrate | acid | mixture |
| Example | (mg) | (mg) | (mg) | (mg) | (% m/m) |
| 1 | 10 | 10 | 10 | 3 | 20/80 |
| 2 | 10 | 10 | 10 | 3 | 50/50 |
| 3 | 1.0 | 5 | — | 3 | 70/30 |
| 4 | 100 | — | 10 | 5 | 70/30 |
| 5 | 10 | — | 20 | 2 | 70/30 |
| 6 | 1.0 | — | 10 | 3 | 90/10 |
| 7 | 0.5 | — | 10 | 2 | 90/10 |
| 8 | 1000 | — | — | 4 | 90/10 |
| 9 | 100 | — | — | 3 | 90/10 |
| 10 | 10 | — | — | 4 | 95/5 |
| 11 | 2.5 | — | — | 3 | 95/5 |
| 12 | 0.5 | 5 | — | 3 | 95/5 |
| 13 | 10 | — | 5 | 3 | 100/0 |
| 14 | 10 | 5 | — | 3 | 100/0 |

H) The Table that follows shows examples of formulations of the R-enantiomer of the compound of Example 1:

100 ml of pharmaceutical preparation contain:

| | | | | | to 100 ml |
|---|---|---|---|---|---|
| | | | | | with |
| | | | disodium | | ethanol/ |
| | 1 | benzalkonium | edetate | citric | water |
| Ex- | (1′-CH₃SO₃H) | chloride | dihydrate | acid | mixture |
| ample | (mg) | (mg) | (mg) | (mg) | (% m/m) |
| 1 | 10 | 10 | 10 | 3 | 20/80 |
| 2 | 10 | 10 | 10 | 3 | 50/50 |
| 3 | 1.0 | 5 | — | 3 | 70/30 |
| 4 | 100 | — | 10 | 5 | 70/30 |
| 5 | 10 | — | 20 | 2 | 70/30 |
| 6 | 1.0 | — | 10 | 3 | 90/10 |
| 7 | 0.5 | — | 10 | 2 | 90/10 |
| 8 | 1000 | — | — | 4 | 90/10 |
| 9 | 100 | — | — | 3 | 90/10 |
| 10 | 10 | — | — | 4 | 95/5 |
| 11 | 2.5 | — | — | 3 | 95/5 |
| 12 | 0.5 | 5 | — | 3 | 95/5 |
| 13 | 10 | — | 5 | 3 | 100/0 |
| 14 | 10 | 5 | — | 3 | 100/0 |

I) The Table that follows shows examples of formulations of the R-enantiomer of the compound of Example 17:

100 ml of pharmaceutical preparation contain:

| | | | | | to 100 ml |
|---|---|---|---|---|---|
| | | | | | with |
| | | | disodium | | ethanol/ |
| | 1 | benzalkonium | edetate | citric | water |
| Ex- | (1′-CF₃COOH) | chloride | dihydrate | acid | mixture |
| ample | (mg) | (mg) | (mg) | (mg) | (% m/m) |
| 1 | 10 | 10 | 10 | 3 | 20/80 |
| 2 | 10 | 10 | 10 | 3 | 50/50 |
| 3 | 1.0 | 5 | — | 3 | 70/30 |
| 4 | 100 | — | 10 | 5 | 70/30 |
| 5 | 10 | — | 20 | 2 | 70/30 |
| 6 | 1.0 | — | 10 | 3 | 90/10 |
| 7 | 0.5 | — | 10 | 2 | 90/10 |
| 8 | 1000 | — | — | 4 | 90/10 |
| 9 | 100 | — | — | 3 | 90/10 |
| 10 | 10 | — | — | 4 | 95/5 |
| 11 | 2.5 | — | — | 3 | 95/5 |
| 12 | 0.5 | 5 | — | 3 | 95/5 |
| 13 | 10 | — | 5 | 3 | 100/0 |
| 14 | 10 | 5 | — | 3 | 100/0 |

J) The Table that follows shows examples of formulations of the R-enantiomer of the compound of Example 13:

100 ml of pharmaceutical preparation contain:

| | | | disodium | | to 100 ml with |
|---|---|---|---|---|---|
| | 1 | benzalkonium | edetate | citric | ethanol/water |
| | (1′-HCl) | chloride | dihydrate | acid | mixture |
| Example | (mg) | (mg) | (mg) | (mg) | (% m/m) |
| 1 | 10 | 10 | 10 | 3 | 20/80 |
| 2 | 10 | 10 | 10 | 3 | 50/50 |
| 3 | 1.0 | 5 | — | 3 | 70/30 |
| 4 | 100 | — | 10 | 5 | 70/30 |
| 5 | 10 | — | 20 | 2 | 70/30 |
| 6 | 1.0 | — | 10 | 3 | 90/10 |
| 7 | 0.5 | — | 10 | 2 | 90/10 |
| 8 | 1000 | — | — | 4 | 90/10 |
| 9 | 100 | — | — | 3 | 90/10 |
| 10 | 10 | — | — | 4 | 95/5 |
| 11 | 2.5 | — | — | 3 | 95/5 |
| 12 | 0.5 | 5 | — | 3 | 95/5 |
| 13 | 10 | — | 5 | 3 | 100/0 |
| 14 | 10 | 5 | — | 3 | 100/0 |

1. A pharmaceutical formulation comprising as sole active substance one or more compounds of formula 1 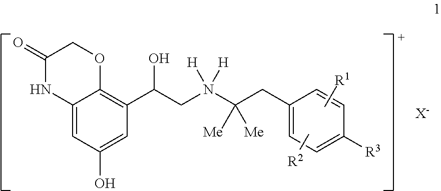
wherein
R¹ is hydrogen, C₁-C₄-alkyl, C₁-C₄-alkoxy or halogen;
R² is hydrogen, C₁-C₄-alkyl, C₁-C₄-alkoxy or halogen;
R³ is hydrogen, C₁-C₄-alkyl, C₁-C₄-alkoxy, halogen, OH, —O—C₁-C₄-alkylene-COOH or —O—C₁-C₄-alkylene-COO—C₁-C₄-alkyl,
X⁻ is an anion with a single negative charge, preferably an anion with a single negative charge selected from the group consisting of chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and
p-toluenesulphonate,
optionally in the form of their tautomers, enantiomers, mixtures of enantiomers, racemates or solvates,
at least one pharmacologically acceptable acid, optionally other pharmacologically acceptable excipients and/or complexing agents and, as solvent, water, ethanol or a mixture of water and ethanol.

2. The pharmaceutical formulation according to claim 1, characterised in that it contains one or more compounds of formula 1, wherein
R¹ is hydrogen, methyl, ethyl, fluorine or chlorine;
R² is hydrogen, methyl, ethyl, fluorine or chlorine;
R³ is hydrogen, methyl, ethyl, propyl, OH, methoxy, ethoxy, fluorine, chlorine, bromine, —O—CH₂—COOH, —O—CH₂—COOmethyl or —O—CH₂—COOethyl, —O—CH₂—CH₂COOH, —O—CH₂—CH₂COOmethyl or —O—CH₂—CH₂COOethyl, CH₂—CH₂—CH₂COOH, —O—CH₂—CH₂—CH₂COOmethyl or —O—CH₂—CH₂—CH₂COOethyl;
X⁻ is an anion with a single negative charge wherein the anion with a single negative charge is selected from the group consisting of chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and
p-toluenesulphonate,
optionally in the form of their tautomers, enantiomers, mixtures of enantiomers, racemates or solvates.

3. The pharmaceutical formulation according to claim 1, characterised in that it contains one or more compounds of formula 1, wherein
R¹ is hydrogen or methyl;
R² is hydrogen or methyl;
R³ is methyl, OH, methoxy, fluorine, chlorine, bromine, —O—CH₂—COOH or —O—CH₂—COOethyl;
X⁻ is an anion with a single negative charge selected from the group consisting of chloride, bromide, sulphate, methanesulphonate, maleate, acetate, benzoate, citrate, salicylate, trifluoroacetate, fumarate, tartrate and succinate;
optionally in the form of their tautomers, enantiomers, mixtures of enantiomers, racemates or solvates.

4. The pharmaceutical formulation according to claim 1, wherein the pharmacologically acceptable acid is selected from inorganic acids hydrochloric acid, hydrobromic acid, nitric acid, sulphuric acid and phosphoric acid or from the organic acids ascorbic acid, citric acid, malic acid, tartaric acid, maleic acid, succinic acid, fumaric acid, acetic acid, formic acid and propionic acid.

5. The pharmaceutical formulation according to claim 1, characterised by a pH of 2.5 to 6.5.

6. The pharmaceutical formulation according to claim 1, characterised in that it contains benzalkonium chloride as excipient.

7. The pharmaceutical formulation according to claim 6, characterised in that the content of benzalkonium chloride is 1 to 50 mg per 100 ml solution.

8. The pharmaceutical formulation according to claim 1, characterised in that the content of formula 1′ is about 0.1 to 1600 mg per 100 ml solution.

9. The pharmaceutical formulation according to claim 1, characterised in that it contains a complexing agent as a further ingredient.

10. The pharmaceutical formulation according to claim 9, characterised in that the content of complexing agent is 1 to 50 mg per 100 ml solution.

11. The pharmaceutical formulation according to claim 1, characterised in that it contains water as solvent.

12. The pharmaceutical formulation according to claim 1, characterised in that it contains ethanol as solvent.

13. The pharmaceutical formulation according to claim 1, characterised in that it contains a mixture of water and ethanol as solvent.

14. The pharmaceutical formulation according to claim 13, characterised in that it contains as solvent a mixture of water and ethanol, wherein the percentage proportion of ethanol by mass is in the range from 5 to 99% ethanol.

15. A pharmaceutical formulation containing as sole active substance a free base of formula 1′ 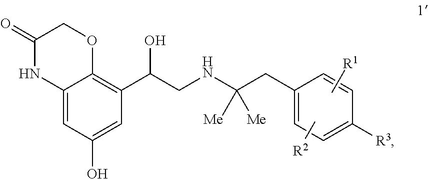
wherein the groups R¹, R² and R³ may have the meanings given in claim 1, optionally in the form of their tautomers, enantiomers, mixtures of enantiomers, racemates or solvates,
at least one pharmacologically acceptable acid, optionally other pharmacologically acceptable excipients and/or complexing agents
and, as solvent, water, ethanol or a mixture of water and ethanol.

16. A pharmaceutical composition comprising a compound according to claim 1 or claim 15 and a pharmaceutically acceptable carrier or excipient thereof.

17. An inhalation kit comprising a pharmaceutical formulation according to claim 1 or claim 15 and an inhaler suitable for nebulising said pharmaceutical formulation.

18. (canceled)
